(19) United States
(12) Patent Application Publication
(10) Pub. No.: US 2008/0091261 A1
(43) Pub. Date: Apr. 17, 2008

(54) IMPLANTABLE VALVE PROSTHESIS

(75) Inventors: Andrew Wallays Long, New York, NY (US); Attila Csordas, Omaha, NE (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: CREIGHTON UNIVERSITY, Omaha, NE (US)

(21) Appl. No.: 11/872,504

(22) Filed: Oct. 15, 2007

Related U.S. Application Data
(60) Provisional application No. 60/851,368, filed on Oct. 13, 2006.

(51) Int. Cl. A61F 2/06

(52) U.S. Cl. 623/1.24

(57) ABSTRACT

An implantable valve prosthesis (10) having a deformable body (12) defining an upstream opening in fluid communication with a downstream opening wherein the deformable body (12) has a first configuration that permits fluid flow in one direction only and a second configuration that prevents retrograde fluid flow in the opposite direction is disclosed.

### CROSS-REFERENCE TO RELATED APPLICATION

This patent application claims priority from U.S. provisional patent application Ser. No. 60/851,368 filed on Oct. 13, 2006 and is herein incorporated by reference in its entirety.

### BACKGROUND

In human pathology, the proper functioning of both cardiac and venous valves is of paramount importance. Tricuspid valves (having three leaflets) are found in the heart and enable the heart to act as a pump by allowing only unidirectional flow of blood. The heart valves are also subject to various disorders such as mitral stenosis, mitral regurgitation, aortic stenosis, aortic regurgitation, mitral valve prolapse and tricuspid stenosis. These disorders are serious and potentially life threatening and may be treated by surgical replacement of the deficient valve.

The veins of the human circulatory system have one-way bicuspid valves comprising two leaflets which promote the flow of blood from the extremities back to the heart by preventing the retrograde flow of blood to the extremities between heart beats. The presence of the venous valves also allows muscular action to assist in the pumping of blood from the venous side of the circulatory system back to the heart. The contraction of skeletal muscles tends to constrict the veins, forcing blood to flow, and the venous valves facilitate the one-way flow of the low-pressure venous blood back to the heart.

Veins are subject to various disorders related to defective structure and function of their valves, known as valve incompetence. Valve incompetence can cause varicose veins, as well as chronic venous insufficiency, in which the valve leaflets become thickened and contracted, thereby rendering the valves incapable of preventing the retrograde flow of blood. Both varicose veins and chronic venous insufficiency cause considerable discomfort and can lead to further complications such as edema, erythema, dermatitis, skin ulceration and cellulitis.

Chronic venous insufficiency (CVI) of the lower extremities is a common condition in the United States; over 2 million new cases of venous thrombosis are recorded each year and about 800,000 new cases of venous insufficiency syndrome will also be recorded annually in the United States. Studies have indicated that about 40% of seriously affected individuals cannot work or travel outside the home and approximately two million workdays are lost each year in the United States as a direct result of CVI.

Numerous therapies have been advanced to treat the symptoms of vericose veins and CVI, and to correct incompetent valves. Less invasive procedures include compression, elevation and wound care, but these treatments tend to be somewhat expensive and are not curative. Surgical interventions may be used to repair, reconstruct or replace the incompetent or damaged valves. Surgical procedures include valvuloplasty (valve repair), valve transplantation, and transposition of veins, all of which provide somewhat limited results. The leaflets of venous valves are generally thin, and once a venous valve becomes incompetent or destroyed, any repair provides only marginal relief. As an alternative to surgical intervention, drug therapy to correct valvular incompetence has been attempted, with limited effectiveness. Other means and methods for treating and/or correcting damaged or incompetent valves include utilizing xenograft valve transplantation (monocusp bovine pericardium), prosthetic or bioprosthetic vascular grafts, and prosthetic venous valves.

The prosthetic venous valves currently available may be categorized as biologic valves or mechanical valves, based on their material of construction and the rigidity of the leaves of the valves. Biologic valves are usually comprised of a stent supporting a number of circumferential leaflets made of a flexible material, or a ring of flexible material attached to two or more circumferential leaflets made of a flexible material. The biological material used in the construction of the valve may be harvested from a human or non-human cadaver. For example, human pericardium biological tissue has been utilized as a covering to stent implants as well as providing the valve leaflets. In addition, non-biologic material such as polyurethane has also been used in the construction of biologic prosthetic valves. Mechanical valves usually comprise a rigid annulus supporting at least two rigid leaflets. The annulus and leaflets are often formed from pyrolitic carbon, a particularly hard and wear resistant form of carbon. The annulus is often situated within a sewing ring so that the valve may be attached to tissue at the location of the replaced valve.

The placement of prosthetic venous valves may be done using surgical implantation or alternatively using minimally invasive techniques. Surgically positioning these implants typically requires suturing or sewing the device into the blood vessel, increasing the risk of thrombosis due to the resulting suturing or anastomoses of the body vessel. Minimally invasive techniques and instruments for placement of intraluminal medical devices have gained widespread use, and coronary and peripheral stents have proven to be a superior means of maintaining vessel patency. A number of existing prosthetic venous valves incorporate a stent in the design, in part to facilitate the placement of the valves using minimally invasive techniques. While the use of stents in the design of a venous valve may eliminate many of the problems associated with invasive surgical implantation techniques, the incorporation of a rigid stent support in the design of a venous valve raises another host of issues.

Venous valves with a stent support element can reduce the effective orifice area of the valve, resulting in a detrimental increase in the transvalvular pressure gradient. A further drawback to a stent valve design is that the stent has fixed dimensions and remains in contact with the total circumference of the inner venous surface and may irritate a large amount of the venous wall, in particular the endothelium, ultimately resulting in intimal hyperplasia and thrombosis. In addition, because the venous diameter normally fluctuates, but the stent does not change dimension, further trauma to the wall of the vein may be induced by the resulting shear stress between the venous wall and the stent. Lastly, the rigidity of the stent support of stent valves compromises the function of the skeletal muscles surrounding the peripheral veins that compress the veins and impel the flow of blood back to the heart.

Another challenging problem that exists with all prosthetic valves currently available, regardless of design, is the tendency to develop thrombosis due to the accrual of biomaterial around the valve elements. The leaves of the valve tend to shelter a small downstream area from the blood flow, creating a region in which biomaterial can accrue, gradually degrading the function of the valve and ultimately contributing to thrombitic formation. Previous designs have incorporated specific coatings or materials on the leaves of the valves to inhibit the accrual of biomaterial, or have allowed a limited amount of backflow either through the incorporation of perforations in the leaves of the valve or leaflet shapes that do not seal completely. The designs of prosthetic valves to date have met with limited success with respect to the inhibition of the accretion of biomaterial.

A continuing need exists, therefore, for improvements in valve replacement systems and in methods for placement and securing of prosthetic valves. Prosthetic valves for the replacement of incompetent venous valves or diseased heart valves should be bio-compatible, long-lasting, structurally compatible with the surrounding vessel walls, and should have the appropriate hemodynamic characteristics which approximate those of natural valves to properly control and promote the flow of blood throughout the circulatory system.

The art has seen several attempts for providing a prosthetic valve to alleviate the consequences of cardiac valve disorders and of venous insufficiency. These attempts generally fall into two categories, biologic valves and mechanical valves. Biologic valves are comprised of a stent supporting a number of circumferential leaflets made of a flexible material. If the material is biologic in nature, it may be either a xenograft, that is, harvested from a non-human cadaver, or an allograft, that is, harvested from a human cadaver. For example, it is known in the art to apply a pericardium biological tissue layer covering, for providing the valve leaflets, to a stent which provides structural annular integrity to the prosthesis. Non-biologic material such as polyurethane has also been used. The second category of prosthetic valves, mechanical valves, usually comprise a rigid annulus supporting up to three rigid leaflets. The annulus and leaflets are frequently formed in pyrolitic carbon, a particularly hard and wear resistant form of carbon. The annulus is captured within a sewing ring so that the valve may be attached to tissue at the location of the replaced valve. Unfortunately, surgically positioning these implants typically requires suturing or sewing the device into the blood vessel, increasing the risk of thrombosis due to the resulting suturing or anastomoses of the body vessel.

These attempts typically provide a valve structure having a relatively rigid tubular body structure which supports a flexible valve leaf structure. That is, any structural rigidity imparted to the tubular body structure is separated from the valve leaf structure. For example, U.S. Pat. No. 4,759,759 discloses a prosthetic valve having a solid stent member having a diametrically-opposed upstanding posts and a substantially cylindrical flexible cover. The two portions of the cover extending between the upstanding stent posts may be collapsed against each other in sealing registry over a fluid passageway defined by the stent. The stent, being a solid member, limits the radial collapsing thereof for endoscopic delivery within a body lumen. The cover, being unsupported by the stent within the fluid passageway of the valve, must itself provide sufficient strength and resiliency to optimally regulate fluid flow. Alternatively, U.S. Pat. No. 5,855,691 discloses a prosthetic valve having a radially expandable covered stent which defines an elongate fluid passageway therethrough. A flexible valve is disposed within the fluid passageway to regulate fluid flow therethrough. The valve is formed of a flexible and compressible material formed into a disc with at least three radial incisions to form deflectable leaflets. While the stent circumferentially supports the valve body, the leaflets are not supported by any other structure within the fluid passageway. Therefore, there exists a need in the art for a unitary prosthetic valve construction that provides structural reinforcement to both the tubular body portion of the valve and to the valve leafs supported thereon.

### SUMMARY

In one embodiment, an implantable valve prosthesis may include a deformable body having a first configuration that permits fluid flow communication in one direction while a second configuration prevents fluid communication in an opposite direction. The deformable body defines a generally cylindrical configuration with a downstream opening in communication with an opposing upstream opening such that when the deformable body is in the first configuration the downstream opening has substantially the same shape as the upstream opening, and when the deformable body is in the second configuration the downstream opening has a smaller shape than the upstream opening, thereby preventing fluid flow communication in the opposite direction.

In an alternative embodiment, an implantable valve prosthesis may include a deformable body having a first position for permitting fluid flow communication inside a lumen in one direction only and a second position for preventing fluid flow communication inside the lumen in the opposite direction with the deformable body being adapted to engage an expandable stent.

In another embodiment, a method for deploying an implantable valve may include the steps of:

providing a catheter defining a proximal end and a distal end;

attaching the distal end of the catheter to an implantable valve prosthesis having a deformable body having a first configuration that permits fluid flow communication in one direction while a second configuration prevents fluid communication in an opposite direction with the deformable body defining a generally cylindrical configuration with a downstream opening in communication with an opposing upstream opening such that when the deformable body is in the first configuration the downstream opening has substantially the same shape as the upstream opening, and when the deformable body is in the second configuration the downstream opening has a smaller shape than the upstream opening, thereby preventing fluid flow communication in the opposite direction; and implanting the distal end of the catheter inside the lumen of a body such that the implantable valve prosthesis is disposed across the lumen of the body in a manner that permits selective fluid flow communication through the lumen by the deformable body of the implantable valve prosthesis.

Additional objectives, advantages and novel features will be set forth in the description which follows or will become apparent to those skilled in the art upon examination of the drawings and detailed description that follows.

### BRIEF DESCRIPTION OF THE DRAWINGS

FIG. 1A is a perspective view of an embodiment of the implantable valve prosthesis having a deformable body that permits fluid flow communication in one direction only through the implantable valve prosthesis;

FIG. 1B is a perspective view of the embodiment of the implantable valve prosthesis shown in FIG. 1A with the deformable body preventing retrograde flow in an opposite direction through the implantable valve prosthesis;

FIG. 2A is a perspective view of another embodiment of the implantable valve prosthesis having a deformable body that permits fluid flow communication in one direction only through the implantable valve;

FIG. 2B is a perspective view of the embodiment of the implantable valve prosthesis shown in FIG. 2A with the deformable body preventing retrograde flow in an opposite direction through the implantable valve prosthesis;

FIG. 3A is a perspective view of an alternative embodiment of the implantable valve prosthesis having a deformable body that permits fluid flow communication in one direction only through the implantable valve;

FIG. 3B is a perspective view of the embodiment of the implantable valve prosthesis shown in FIG. 3A with the deformable body preventing retrograde flow in an opposite direction through the implantable valve prosthesis;

FIG. 4A is a perspective view of yet another embodiment of the implantable valve prosthesis having a deformable body that permits fluid flow communication in one direction only through the implantable valve;

FIG. 4B is a perspective view of the embodiment of the implantable valve prosthesis shown in FIG. 4A with the deformable body preventing retrograde flow in an opposite direction through the implantable valve prosthesis;

FIG. 5A is a perspective view of yet another embodiment of the implantable valve prosthesis having a deformable body that permits fluid flow communication in one direction only through the implantable valve;

FIG. 5B is a perspective view of the embodiment of the implantable valve prosthesis shown in FIG. 5A with the deformable body preventing retrograde flow in an opposite direction through the implantable valve prosthesis;

FIG. 6A is a perspective view of yet another embodiment of the implantable valve prosthesis having a deformable body that permits fluid flow communication in one direction only through the implantable valve;

FIG. 6B is a perspective view of the embodiment of the implantable valve prosthesis shown in FIG. 6A with the deformable body preventing retrograde flow in an opposite direction through the implantable valve prosthesis;

FIG. 7A is a perspective view of yet another embodiment of the implantable valve prosthesis having a deformable body that permits fluid flow communication in one direction only through the implantable valve;

FIG. 7B is a perspective view of the embodiment of the implantable valve prosthesis shown in FIG. 7A with the deformable body preventing retrograde flow in an opposite direction through the implantable valve prosthesis;

FIG. 8 is a perspective view of an embodiment of the implantable valve prosthesis having a deformable body defining a crowned configuration;

FIG. 9 is a perspective view of another embodiment of the implantable valve prosthesis having a deformable body defining a scalloped configuration;

FIG. 10A is a perspective view of yet another embodiment of the implantable valve prosthesis having a deformable body that permits fluid flow communication in one direction only through the implantable valve;

FIG. 10B is a perspective view of the embodiment of the implantable valve prosthesis shown in FIG. 10A with the deformable body preventing retrograde flow in an opposite direction through the implantable valve prosthesis;

FIG. 11 is a partial cross sectional view showing the valve prosthesis implanted inside the lumen of a body with a deformable body in an open position;

FIG. 12 is a cross sectional view showing an opening taken along line 12-12 of FIG. 11;

FIG. 13 is a perspective view of the implantable valve prosthesis shown in FIG. 11 with the deformable body in the open position;

FIG. 14 is a perspective view of the implantable valve prosthesis shown in FIG. 11 with the deformable body in the closed position;

FIG. 15 is a cross-sectional view of the implantable valve prosthesis taken along line 15-15 of FIG. 14 showing the deformable body in the closed position;

FIG. 16 is a partial cross-sectional view of the valve prosthesis implanted within the lumen of a vessel showing the deformable body in the closed position;

FIG. 17 is a perspective view of an embodiment of the implantable valve prosthesis having a cylindrical piece of membrane engaged to one end of the deformable body;

FIG. 18 is a perspective view of another embodiment of the implantable valve prosthesis in which extensions of membrane are added to the valve;

FIG. 19 is a perspective view of an alternate embodiment of the implantable valve prosthesis in which upstream cylinder and downstream extensions have been added to the valve prosthesis;

FIG. 20 is a perspective view of another alternate embodiment of the implantable valve prosthesis in which areas of the valve have been cut out at the sites of attachment to the vessel wall;

FIG. 21 is a perspective view of the same embodiment shown in FIG. 20;

FIG. 22 is a partial cross sectional view of an embodiment of the implantable valve prosthesis shown in FIGS. 20 and 21 in which the valve is implanted in a vessel and in the open position;

FIG. 23 is a cross-sectional view of the implantable valve prosthesis taken along line 23-23 of FIG. 22 showing the downstream opening of the deformable body in the open position;

FIG. 24 is a partial cross-sectional view of an embodiment of the implantable valve prosthesis of FIG. 22 showing the deformable body in the closed position;

FIG. 25 is a cross-sectional view of the implantable valve prosthesis take along line 25-25 of FIG. 24 showing the downstream opening of the deformable body in the closed position;

FIG. 26 is a perspective view of another embodiment of the implantable valve prosthesis with the deformable body having opposing slots defined along the upstream opening of the valve;

FIG. 27 is a perspective view of yet another embodiment of the implantable valve prosthesis with the deformable body having opposing slots defined along both the upstream and downstream openings;

FIG. 28 is a perspective view of an alternate embodiment of the implantable valve prosthesis with the upstream and downstream openings having cuts;

FIG. 29 is a perspective view of the embodiment shown in FIG. 28 with the shaded areas illustrating the areas of attachment between the valve and the vessel;

FIG. 30 is a perspective view of an alternate embodiment of the implantable valve prosthesis in which a cylindrical piece of membrane is added to the upstream opening of the valve;

FIG. 31 is a perspective view of another embodiment of the implantable valve prosthesis in which a cylindrical piece of membrane has been added to the upstream opening of the valve and areas of the upstream and downstream openings have been cut;

FIG. 32 is a perspective view of an embodiment of the implantable valve prosthesis illustrating one method for endoluminal implantation of the valve inside a vessel;

FIG. 33 is a cross-sectional view of the implantable valve prosthesis taken along line 33-33 of FIG. 32 showing a plurality of balloons near the upstream aspect of the valve;

FIG. 34 is a perspective view of an alternate embodiment of the implantable valve prosthesis with the downstream opening shown in the open position in which a conical valve is attached to a conduit;

FIG. 35 is a perspective view of another alternate embodiment of the implantable valve prosthesis with the downstream opening shown in the closed position in which a conical valve is attached to a conduit;

FIG. 36 is a perspective view of another embodiment of the implantable valve prosthesis in which a conical valve is attached to a conduit defining a complete sinus;

FIG. 37 is a perspective view of the embodiment shown in FIG. 36 showing the downstream opening in the closed position;

FIG. 38 is a perspective view of the generally conical deformable body used within the conduit with a complete sinus shown in FIG. 36 with the upstream opening having a larger diameter than the downstream opening;

FIG. 39 is a perspective view of an alternate embodiment of the implantable valve prosthesis;

FIG. 40 is a perspective view of another embodiment of the implantable valve prosthesis with the valve implanted inside a conduit consisting of a full sinus only without a straight tubular portion;

FIG. 41 is a perspective view of a generally conical membrane used with a conduit having a half sinus with the downstream opening shown in the open position;

FIG. 42 is a perspective view of the generally conical membrane of FIG. 41 with the downstream opening shown in the closed position;

FIG. 43 is a perspective view of an embodiment of the implantable valve prosthesis in which the valve is implanted inside a conduit with the conduit consisting of a fully sinus only without a straight tubular portion;

FIG. 44 is a perspective view of the embodiment shown in FIG. 36 illustrating a self-expanding coil-shaped stent attached to the outside surface of the sinus to assist in maintaining the sinus within the vessel;

FIG. 45 is a perspective view of another embodiment of the implantable valve prosthesis with the deformable body having a downstream opening in the open position as well as an expandable stent consisting of two cylindrical regions joined by two opposing longitudinal struts;

FIG. 46 is a perspective view of the implantable valve prosthesis of FIG. 45 with the downstream opening of the valve shown in the closed position;

FIG. 47 is a perspective view of the implantable valve prosthesis in which two separate pieces of flexible membrane are used to form the valve with each piece of flexible membrane being one half of a conical shape with the wall bowed outwardly;

FIG. 48 is a perspective view of two separate pieces of flexible membrane used to form the valve of FIG. 47 with the shaded areas representing the portions of visible surfaces of the membrane pieces which will be attached to the inner surface of the conduit;

FIG. 49 is a perspective view of two separate pieces of flexible membrane which also could be used to form a valve within a conduit;

FIG. 50 is a perspective view of an alternate embodiment of the implantable valve prosthesis in which a generally conical deformable body membrane is attached to a circumferential, balloon expandable, self-expandable frame with the valve in the expanded state and the downstream opening in the open position;

FIG. 51 is a perspective view of the embodiment shown in FIG. 50 in which the generally conical deformable body is attached to a circumferential, balloon expandable or self-expandable frame with the valve in the expanded state and the downstream opening in the closed position;

FIG. 52 is a perspective view of another embodiment of the implantable valve prosthesis having opposing triangular-shaped slots cut out of the downstream opening with a circumferential, balloon expandable or self-expandable frame with the valve in the expanded state and the downstream opening in the open position; and

FIG. 53 is a perspective view of the embodiment shown in FIG. 52 having opposing triangular-shaped slots cut out of the downstream opening with a circumferential, balloon expandable or self-expandable frame with the valve in the expanded state and the downstream opening in the closed position.

Corresponding reference characters indicate corresponding elements among the several views. The headings used in the figures should not be interpreted to limit the scope of the figures.

### DETAILED DESCRIPTION

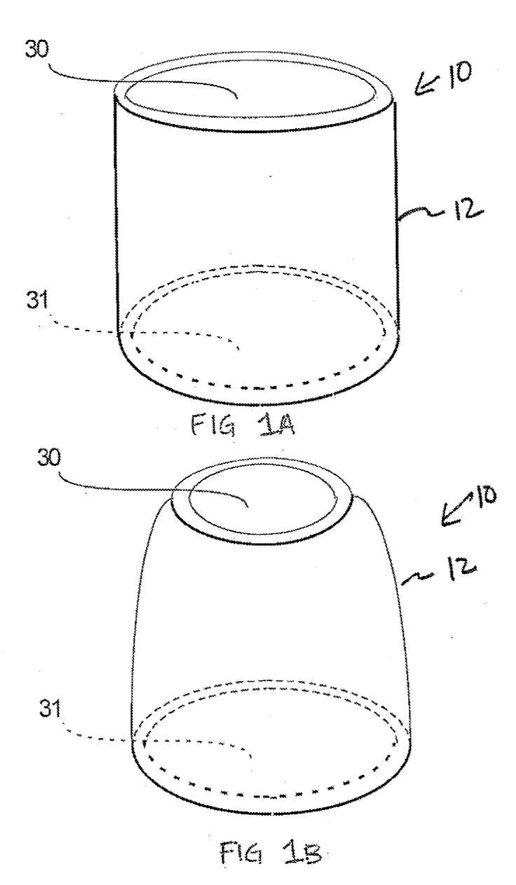

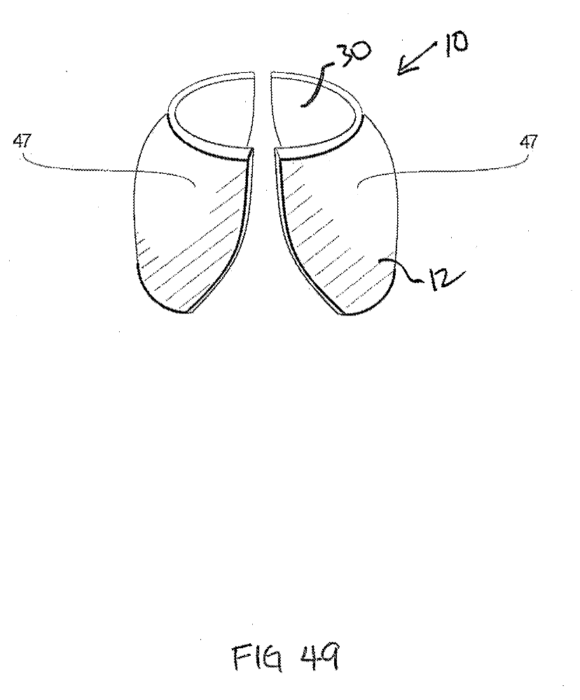

Referring to the drawings, various embodiments of the implantable valve prosthesis are generally indicated as 10 in FIGS. 1-53. The implantable valve prosthesis 10 includes many different embodiments with each embodiment adapted for implantation inside the lumen of a vessel using an open surgical procedure or implanted within the lumen of a vessel using a percutaneous endoluminal catheter in order to make the implantation minimally invasive. In particular, the valve prosthesis 10 may be implanted within any tubular organ or duct of the mammalian body, including the vascular system (e.g., veins), lymphatic system, biliary system, ureters and alimentary tract. Further, the implantable valve prosthesis 10 includes a deformable body 12 defining a downstream opening 30 in fluid flow communication with an upstream opening 31 for controlling the flow of fluid through a body lumen of a vessel 14. The deformable body 12 is operable between an open position that permits fluid flow through the deformable body 12 in one direction only (e.g., from the upstream opening 31 to the downstream opening 30), while preventing retrograde fluid flow in the opposite direction (e.g., from the downstream opening 30 to the upstream opening 31). In one embodiment, the deformable body 12 is reversibly deformable between the open position and the closed position. As used herein, the term “vessel” is used in its most general meaning.

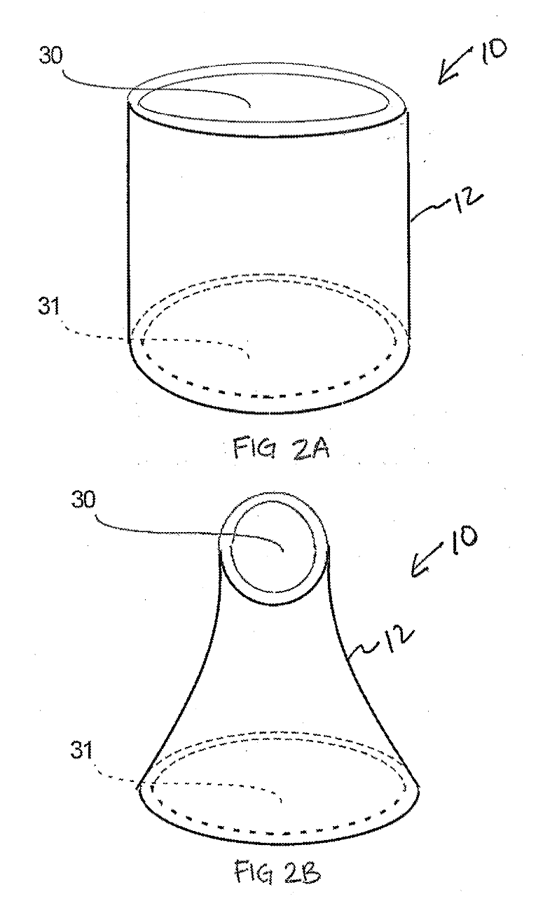

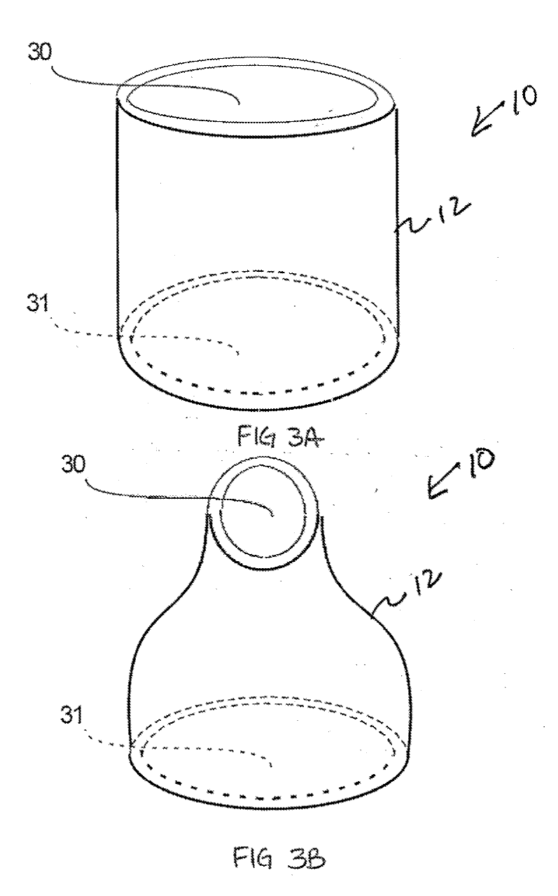

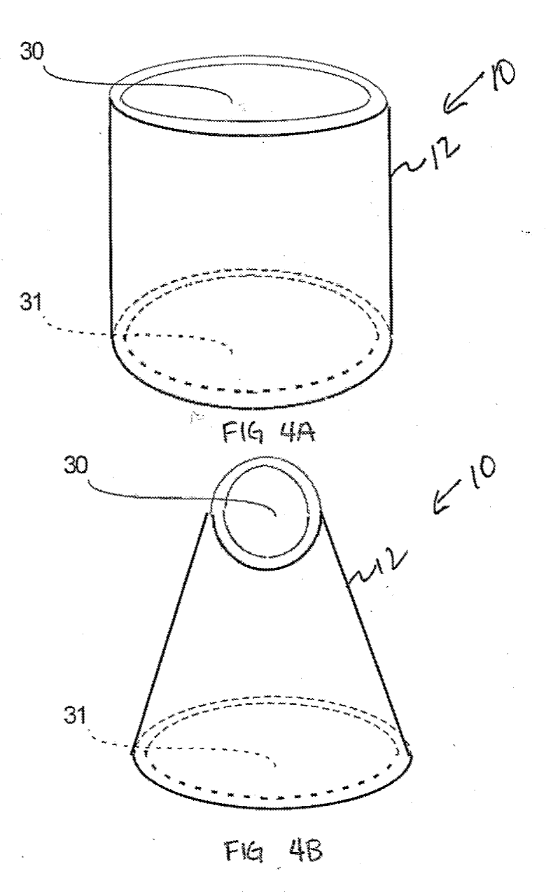

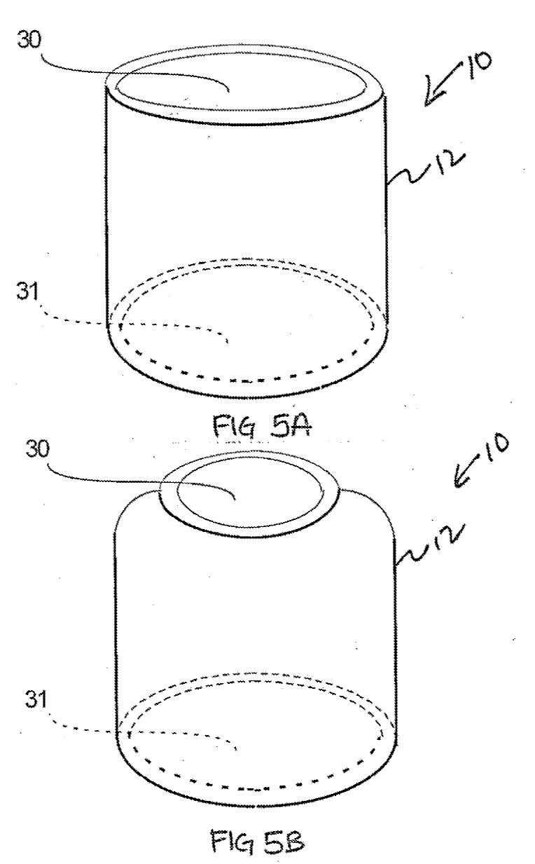

Referring to FIGS. 1A and 1B, an embodiment of the valve prosthesis 10 may include the reversibly deformable body 12 fashioned into a generally conical configuration, with a generally circular upstream opening 31 and a generally elliptical downstream opening 30. The long axis of the elliptical downstream opening 30 is similar or identical in length to the diameter of the circular upstream opening 31. In the embodiment, the walls of the deformable body 12 bow outwards. The valve prosthesis 10 is shown in the open position, which is its default position in a no-flow environment. The bowing outwards of the walls of the deformable body 12 functions in part to minimize the space between the outer surface of the body 12 and the inner surface of the surrounding vessel 14 when the valve prosthesis 10 is the open position, which is advantageous in certain body vessels (such as veins) to minimize the areas of stagnant flow. In alternate embodiments, however, the walls of the deformable body 12 can bow inwards (as depicted in FIGS. 2A and 2B, bow sinusoidally (as depicted in FIGS. 3A and 3B, or be straight (as depicted in FIGS. 4A and 4B). The sinus spaces (spaces between the outer surface of the conical deformable body 12 and the inner surface of the surrounding vessel) can also be minimized with a more cylindrical shape of the body 12, as depicted in FIGS. 5A and 5B. Note that any length of the upstream aspect of the reversibly deformable body 12 can be cylindrical, and the valve prosthesis 10 described herein includes embodiments in which the body 12 is almost entirely cylindrical along its length, with only a minimal portion curving inwards at the downstream end to form an elliptical downstream opening (FIGS. 5A and 5B). Note that the manner in which the cylindrical body 12 narrows to a downstream elliptical opening 30 can be with bowed-out walls, or with bowed-in walls, or sinusoidally bowed walls or with straight walls. The more cylindrical deformable body 12 configurations can be used in all of the other embodiments described herein (please note that the term “generally conical deformable body” as it is used throughout this document also refers to more cylindrical embodiments similar to that depicted in FIGS. 5A and 5B.

The deformable body 12 may be made from a deformable biocompatible material that can be either synthetic or biologic. Possible synthetic materials include, but are not limited to, polytetrafluoroethylene (PTFE), expanded polytetrafluoroethylene (ePTFE), polyurethane, polyethylene (PE), or Dacron, Rayon, and Silicone. Possible biologic materials include, but are not limited to, autologous, allogenic, and xenograft materials. These include explanted veins and decellurized basement membrane materials, such as small intestine submucosa (SIS). The deformable body 12 can be fashioned from a single piece of membrane in a seamless fashion (as depicted in the illustrations), or from a sheet of membrane with a seam that can be secured with sutures, adhesive, or staples. Alternatively, the deformable body 12 can be fashioned from multiple sheets of membrane with multiple seams. The deformable body 12 can be of uniform thickness, or of non-uniform thickness. In addition, the valve prosthesis 10 can be a layered composite of a plurality of synthetic and/or biologic membrane materials. In addition, the deformable body 12 can include a plurality of holes; one purpose of these holes would be to allow a certain degree of retrograde fluid flow which could be advantageous for physiological reasons or to lessen the pressure on the valve caused by retrograde flow.

The deformable body 12 may also be treated and/or coated with any number of surface and/or material treatments. For example, the deformable body 12 can be treated with an anti-thrombogenic material, as are know or will be known. Similarly, the deformable body 12 may be treated with one or more biologically active compounds and/or materials that may promote and/or prevent the in-growth of various cell types (e.g. endothelial cells) onto the membrane. Alternatively, the deformable body 12 may be partially or fully seeded with cultured tissue cells (e.g. endothelial cells) derived from either a donor or the host patient.

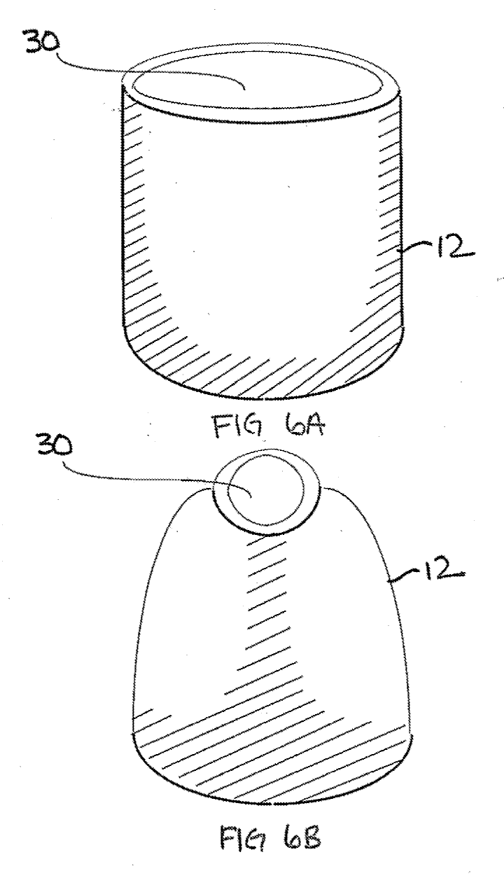

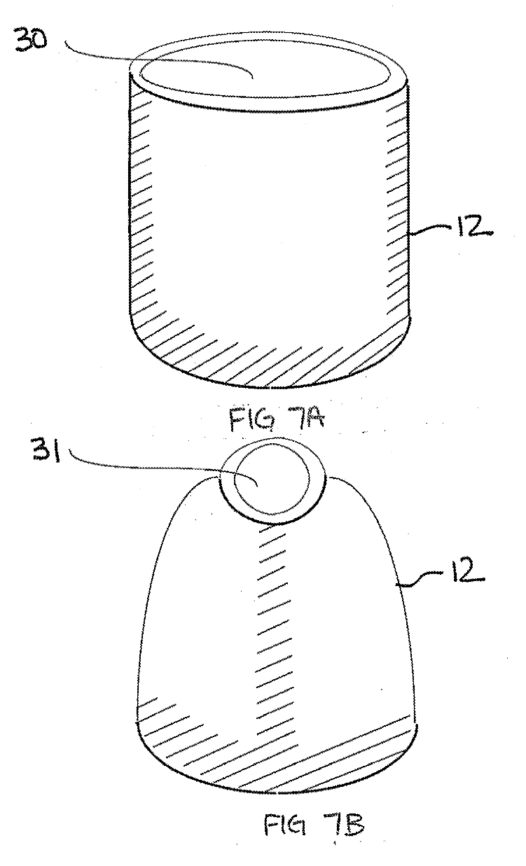

The valve prosthesis 10 is sized for a given lumen so that the diameter of the generally circular upstream opening 31 is approximately the diameter of the vessel lumen at the site of implantation. However, depending on the nature of the vessel 14, the valve prosthesis 10 may need to be oversized or undersized to accommodate for physiologically expected changes in lumen diameter. The outer surface of the generally circular upstream portion of the deformable body 12 is attached to the inner surface of the vessel wall circumferentially, so that the upstream opening 31 more or less maintains its circular shape. However, at the generally elliptical downstream opening 30 only the two opposed areas at either end of the long axis of the ellipse are attached to the inner surface of the vessel wall. The attachment of the deformable body 12 extends from these two opposing areas at the elliptical downstream opening 30 longitudinally to the circumferentially attached upstream area. The areas that remain unattached to the inner surface of the vessel lumen wall will be the valve leaflets. Referring to FIGS. 6A and 6B, an embodiment is depicted demonstrating the area of the outer surface of the generally conical deformable body 12 which will be attached to the inner surface of the vessel wall. This area of attachment defines two opposing, generally parabolic shaped areas of the body 12 which are not attached; these are the valve leaflets. Alternatively, the area of attachment of the generally conical deformable body 12 can define two generally rectangular shaped leaflets, as depicted in FIGS. 7A and 7B. Additional embodiments of this invention can include varying numbers of valve leaflets, including but not limited to one, three, and four leaflet embodiments. The attachment of the deformable body 12 to the inner surface of the body lumen can be achieved with an adhesive substance, including but not limited to heat-activated and UV-activated adhesive substances. Alternatively, sutures and/or staples and/or barbs can be used to achieve this attachment to the vessel wall. In additional embodiments, a balloon-expandable or self-expanding stent frame can be attached to the outer surface of the upstream aspect of the valve prosthesis 10 or incorporated into the upstream aspect of the valve prosthesis 10 or attached to the inner surface of the upstream aspect of the valve prosthesis 10. Alternatively, the generally conical deformable body 12 can be attached to the inner surface of a generally cylindrical stent (as are known or will be known to practitioners of the art), and the stent deployed in the lumen of the intended vessel.

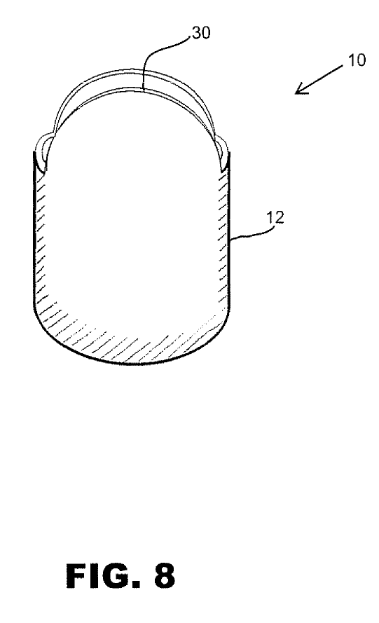

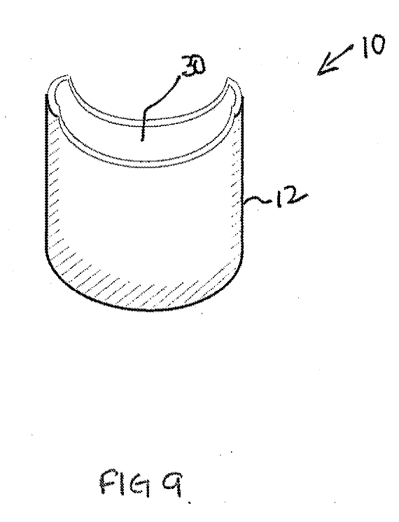

The downstream edge of each leaflet can be flat, as depicted in the original embodiment in FIGS. 6A and 6B. Alternatively, the downstream edge of each leaflet can be crowned, as depicted in FIG. 8, or scalloped, as depicted in FIG. 9. Other shapes of the downstream edges of the leaflets are also considered. The leaflets of a single valve can have identical downstream edges or have different morphologies.

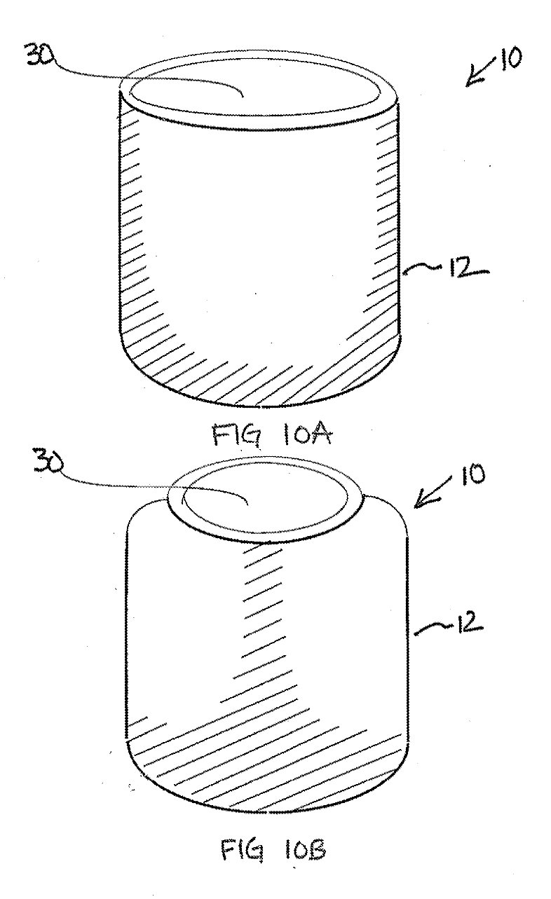

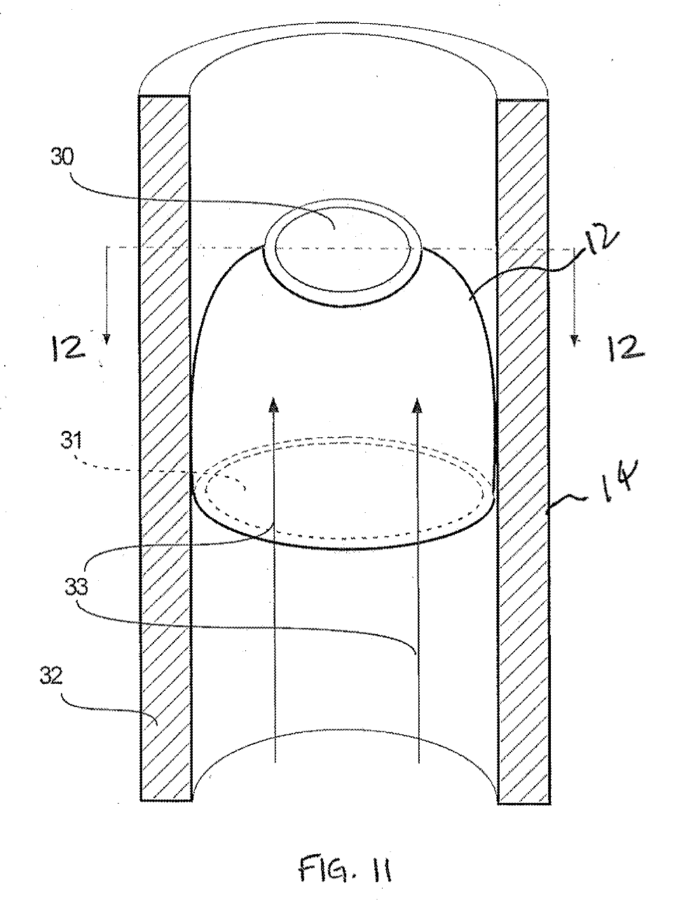

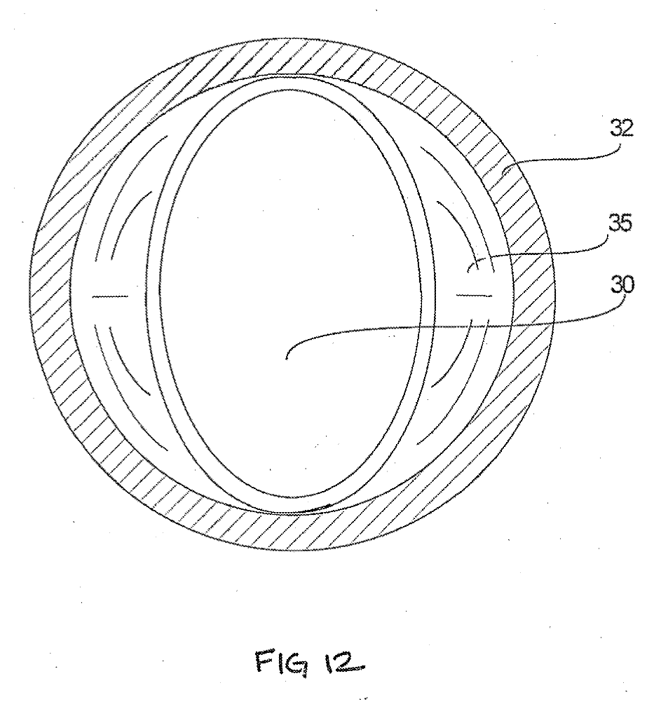

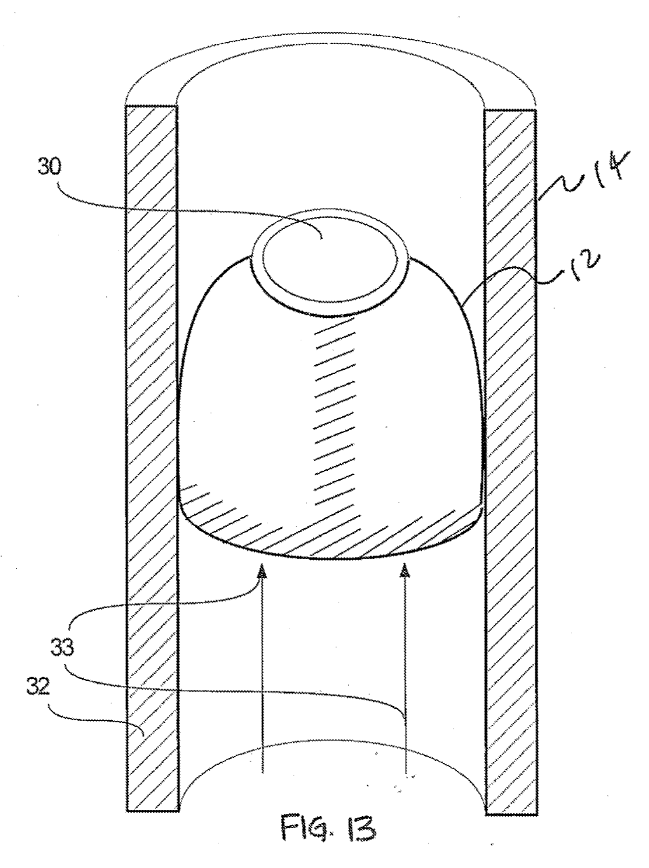

Referring to FIGS. 10A, 10B and 11, the original embodiment is depicted implanted within a body lumen. The valve prosthesis 10 is in the open position, with antegrade fluid flow 33. The area of the outer surface of the deformable body 12 that is attached to the inner surface of the body lumen is depicted in FIG. 13 (the line shaded surface of the deformable body 12 is the portion of the visible surface that will be attached to the inner surface of the vessel wall). Because the downstream aspect of the deformable body 12 is attached the body lumen in two opposing positions, the elliptical downstream opening 30 is maintained when the valve prosthesis 10 is implanted and in the open position (as depicted in FIG. 4). With the valve prosthesis 10 in the open position, the outer surfaces of the valve leaflets 35 are still exposed when viewed from above (as depicted in FIG. 12), so that retrograde flow will cause the valve leaflets to move inward to the closed position.

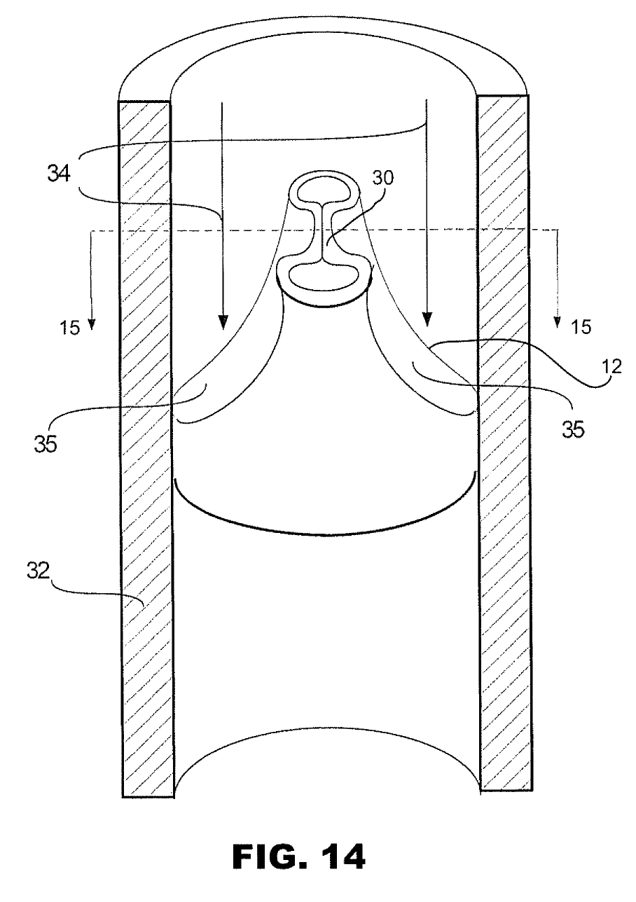

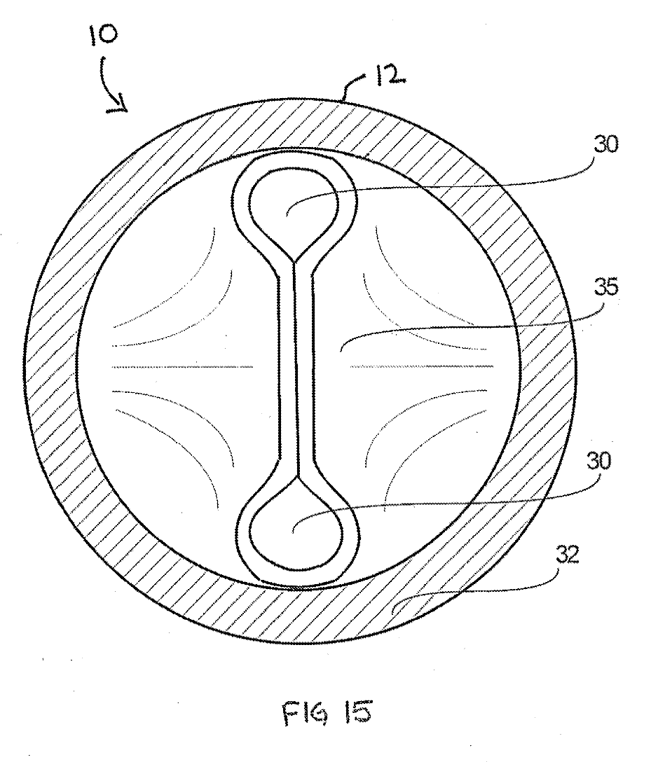

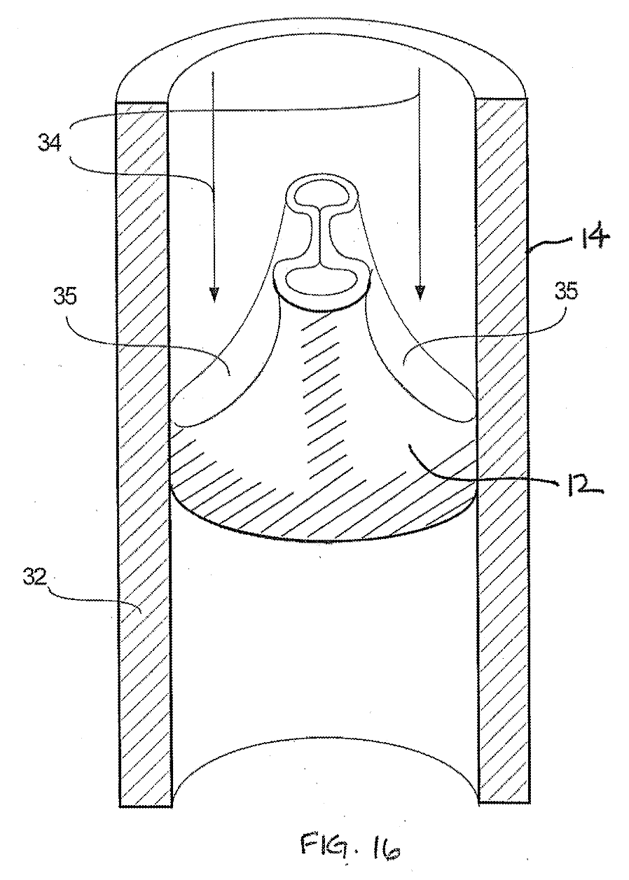

Referring to FIGS. 14 and 16, the embodiment is depicted implanted within a vessel lumen, in the closed position due to retrograde flow 34. Because the downstream aspect of the deformable body 12 is attached to the body lumen in two opposing positions, retrograde flow will act on the outer surfaces of the valve leaflets 35 to close the valve prosthesis 10. With the valve prosthesis 10 in the closed position, the downstream opening 30 of the deformable body 12 is minimized, as depicted in FIG. 15. The valve prosthesis 10 remains in the closed position until pressure on the inner surface of the leaflets by antegrade blood flow inverts the leaflets to the open position.

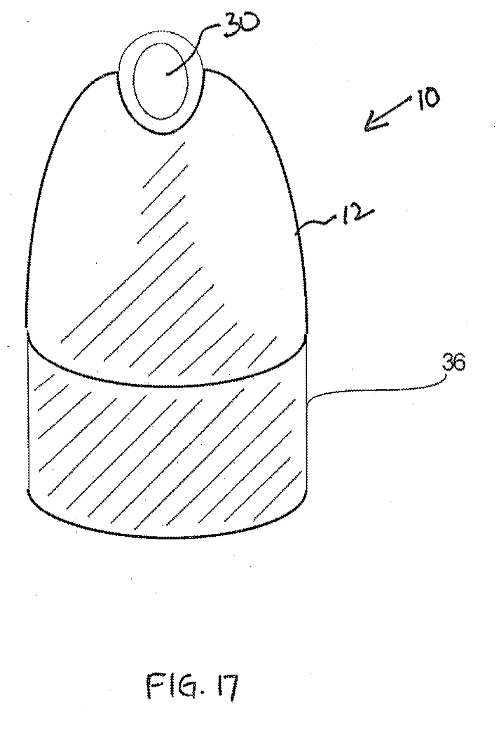

Referring to FIG. 17, an alternative embodiment is depicted in which additional cylindrical membrane material 36 is added to the upstream aspect of the generally conical deformable body 12. This cylindrical deformable body 12 is contiguous with the generally conical membrane, and can be attached to the body lumen circumferentially (as demonstrated by the line shaded area on the surface of the membrane in FIG. 17) to strengthen the attachment of the valve prosthesis 10 to the body lumen. Alternatively, only a small circumferential portion of the upstream aspect of the additional cylindrical membrane material can be attached to the inner surface of the lumen, so that the unattached downstream part of the added cylindrical membrane material functions as part of the valve leaflet. Note the relative lengths of the cylindrical portion of the deformable body 12 and the conical portion of the deformable body 12 can vary, so that the majority of the valve prosthesis 10 is cylindrical with only a short “conical” element at the downstream aspect that converges to form an elliptical downstream opening (as shown in FIGS. 2A, 2B, 10A and 10B).

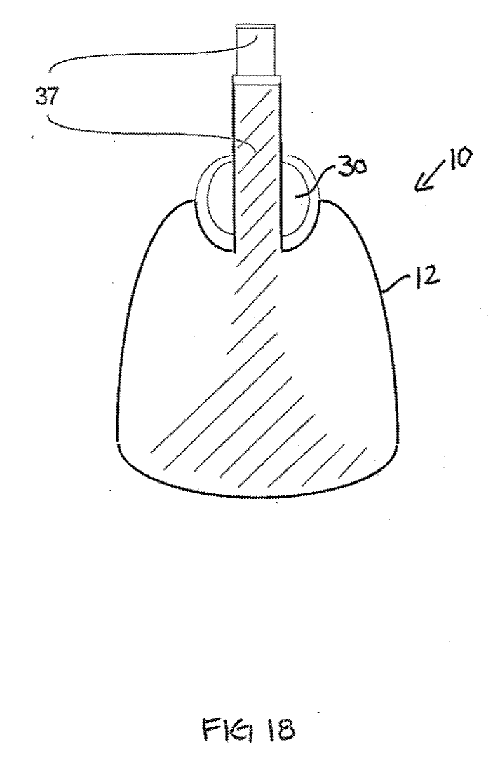

Referring to FIG. 18, an alternative embodiment of the valve prosthesis 10 is depicted in which extensions of membrane 37 are added to the downstream aspect of the generally conical deformable body 12. These extensions are contiguous with the generally conical deformable body 12, and extend the opposing areas of the downstream membrane attachment. The extensions function in part to strengthen the attachment of the valve prosthesis 10 to the body lumen. In valve prosthesis 10 with more than two leaflets, additional extensions can be employed. The extensions can be rectangular, as depicted in FIG. 18, or include an expanded area to further increase the area of attachment to the body lumen.

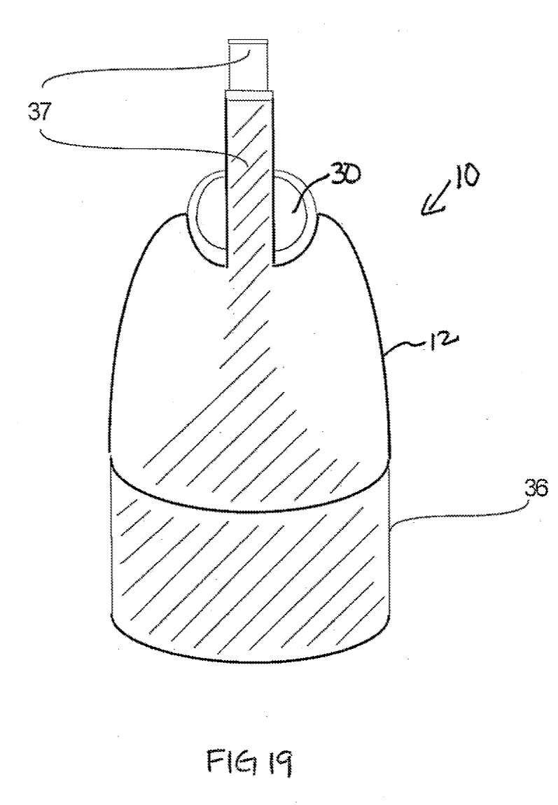

Referring to FIG. 19, an alternate embodiment is depicted in which both extensions of membrane 37 and a cylinder are added to the generally conical deformable body 12.

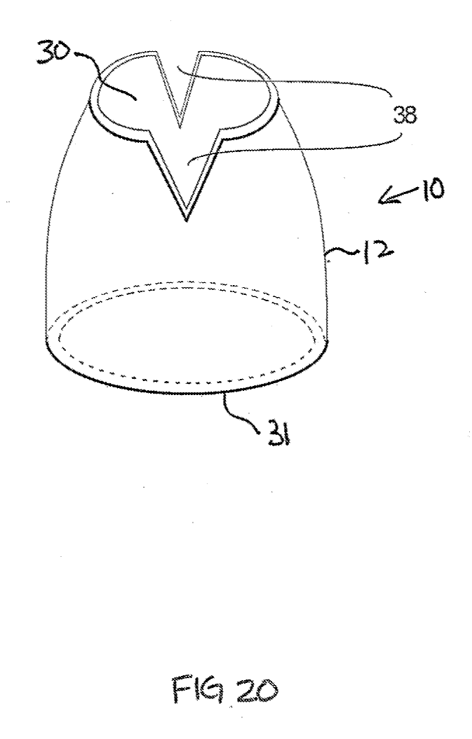

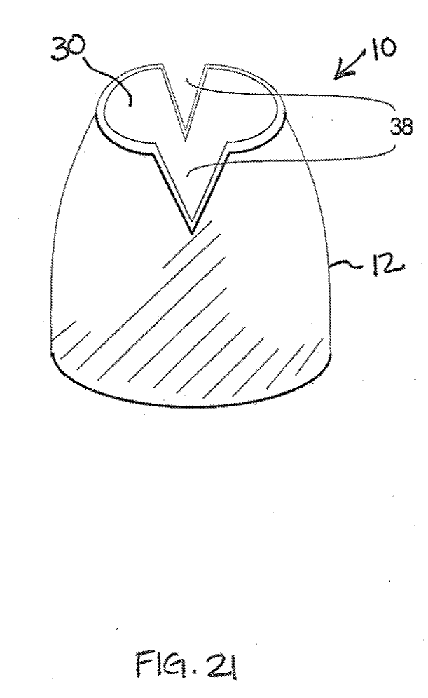

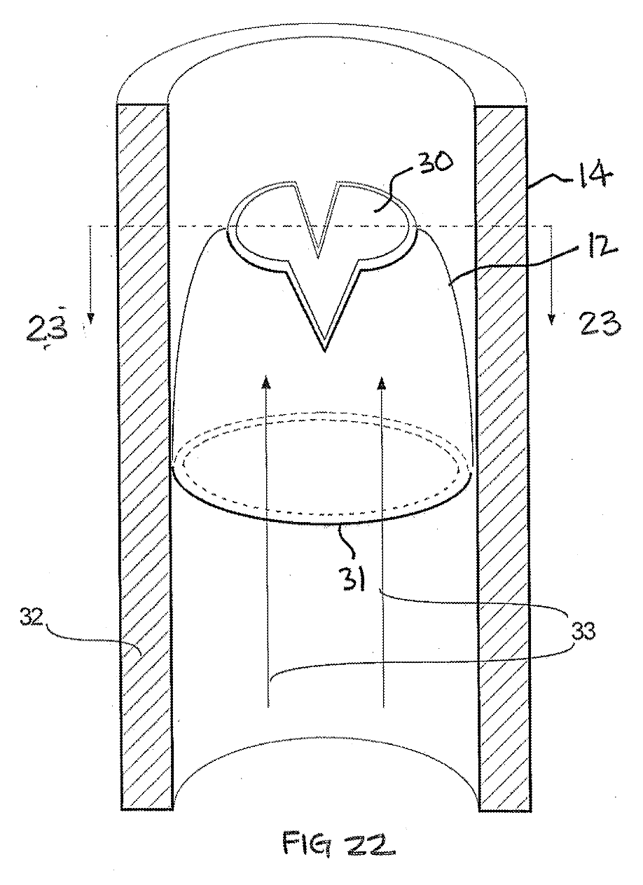

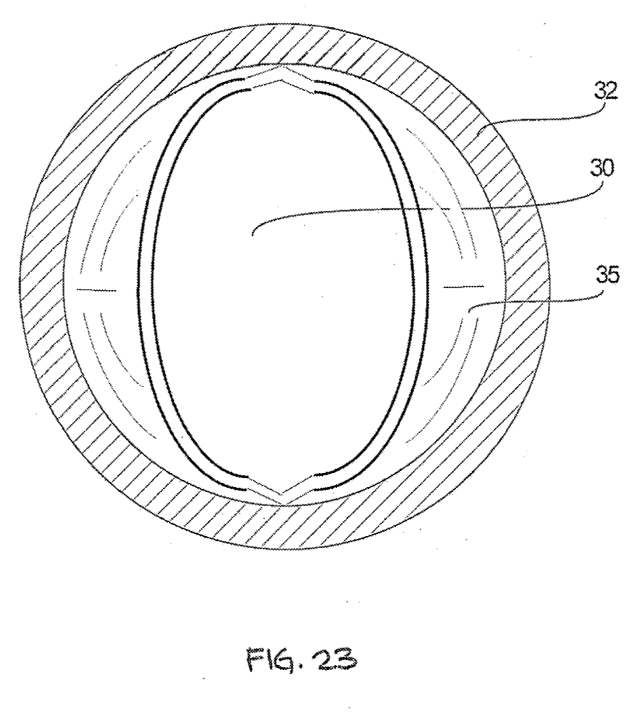

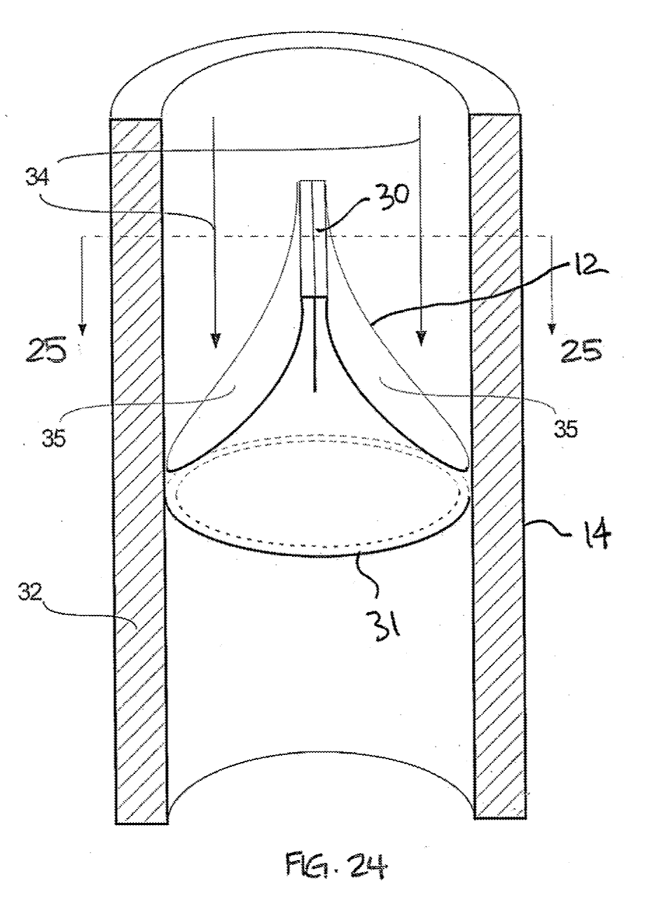

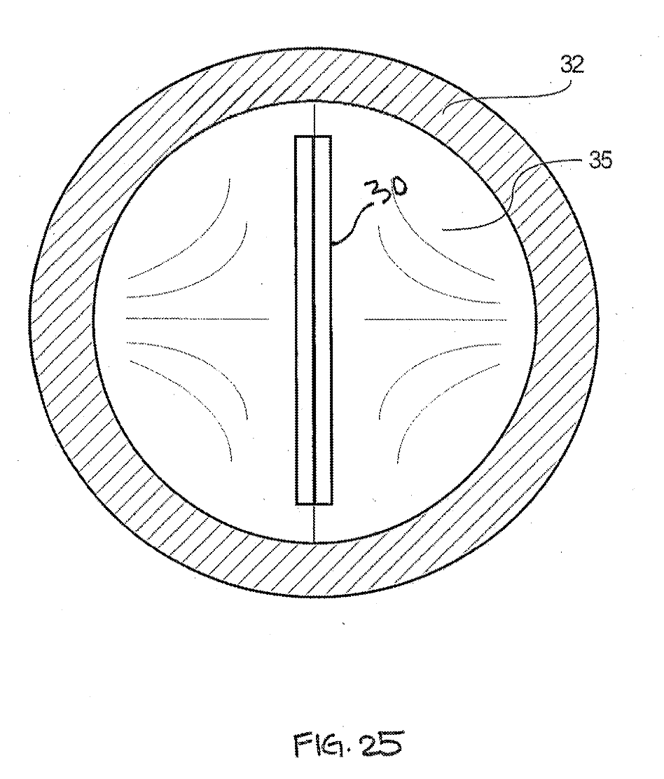

Referring to FIG. 20, an alternate embodiment is depicted in which generally triangle-shaped sections of membrane have been cut out 38 of the downstream aspect of the generally conical deformable body 12. These areas are oriented immediately downstream to the longitudinal sites of attachment between the deformable body 12 and the vessel wall, as depicted in FIG. 21. Similar to the other embodiment, this proximal aspect of this embodiment of the valve prosthesis 10 assumes an open position as depicted in FIGS. 22 and 23 in response to antegrade fluid flow 33 and assumes a closed position as depicted in FIGS. 24 and 25 in response to retrograde fluid flow 34. The advantage of this embodiment is demonstrated when it assumes the closed position depicted in FIGS. 24 and 25. In the closed position, the downstream opening 30 of this embodiment is further reduced relative to the downstream opening 30 of the other embodiment in the closed position, and therefore it is more effective at preventing retrograde flow. The opposing cut out areas in the downstream aspect of the valve prosthesis 10 can be cut in a variety of shapes and sizes with straight and/or curved edges.

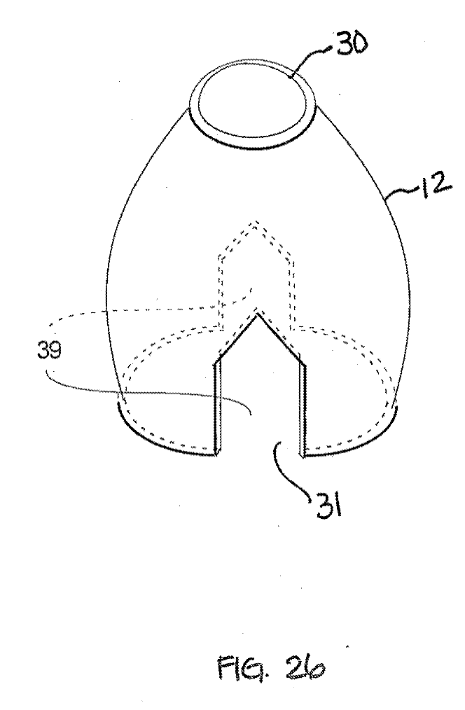

In an additional embodiment depicted in FIG. 26, opposing areas are cut out of the upstream aspect of the generally conical deformable body 12. The advantage of this feature is that it will allow a cylinder of a given diameter to conform to a somewhat smaller vessel, simplifying the sizing of the valve prosthesis 10 for a given vessel. Varying numbers of areas can be cut in the cylindrical portion in a variety of shapes and sizes with straight and/or curved edges.

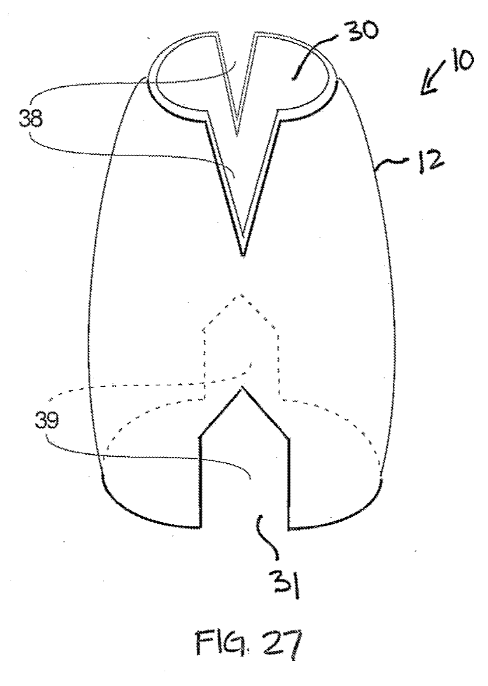

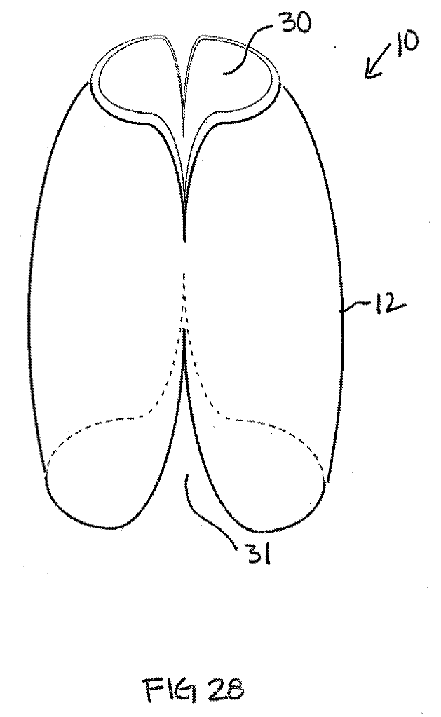

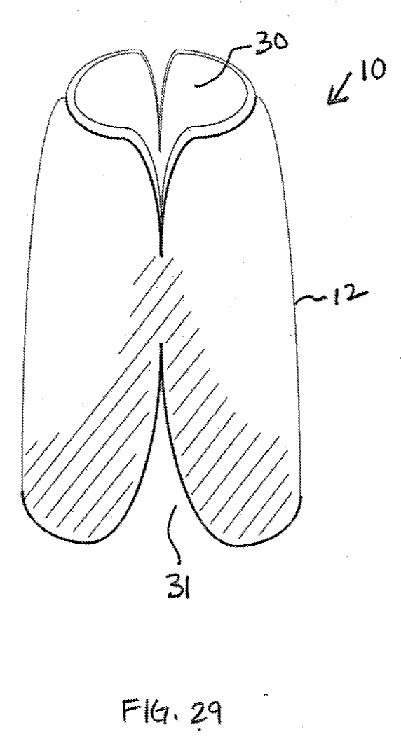

In an additional embodiment depicted in FIG. 27, areas are cut out of both the downstream aspect and the upstream aspect of the generally cylindrical deformable body 12. As previously stated, these areas can be variable in size and shape. FIG. 28 depicts an embodiment with rounded areas cut from the downstream and upstream aspects of the generally conical deformable body 12. FIG. 29 illustrates the area of the outer surface of the embodiment depicted in FIG. 28 which will be attached to the inner surface of the vessel wall.

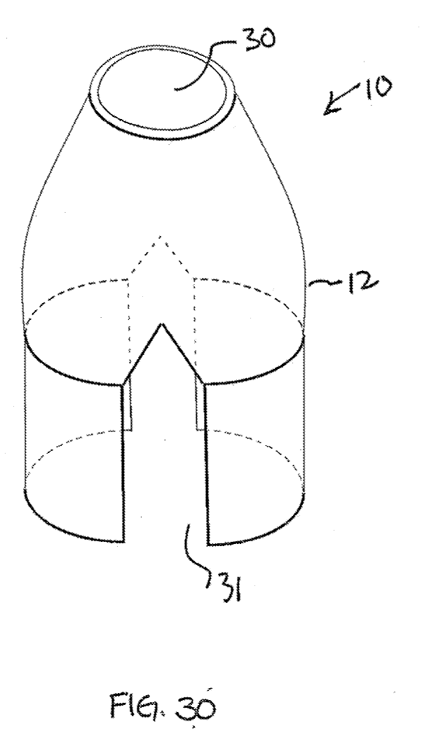

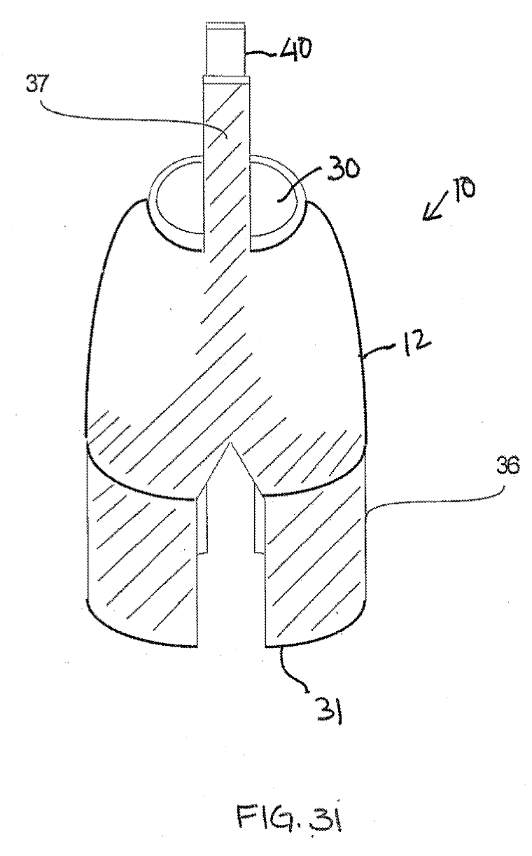

Features of the previously described embodiments can be used in varying combinations within the scope of the invention. For example, FIG. 30 depicts a generally conical deformable body 12 with a contiguous cylindrical section of the deformable body 12 at the upstream aspect, with opposing areas cut out of the upstream aspect. FIG. 31 illustrates the area of the outer surface of the embodiment depicted in FIG. 30 which will be attached to the inner surface of the vessel wall.

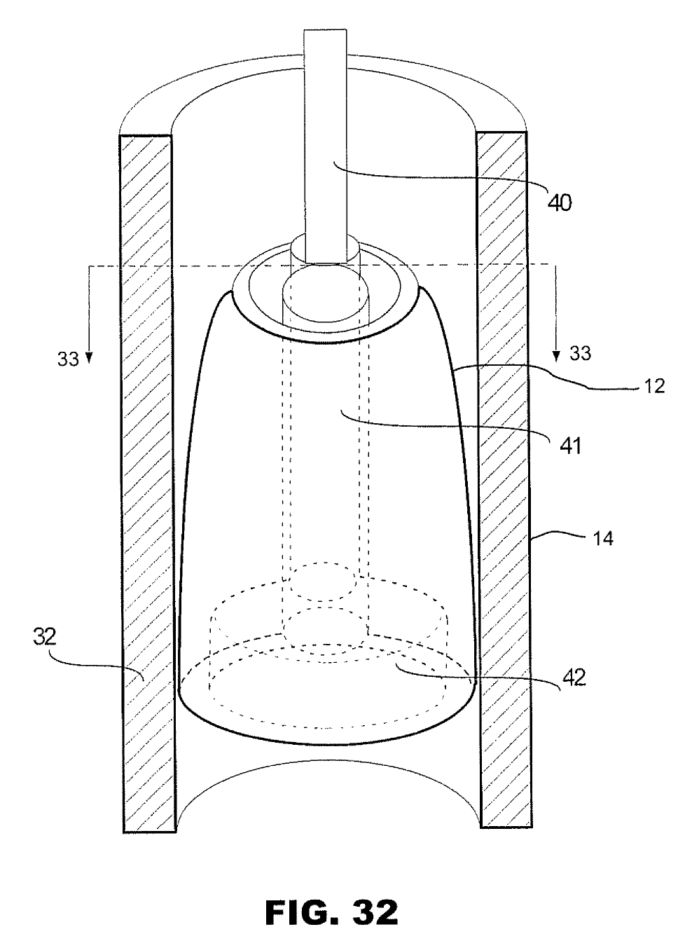

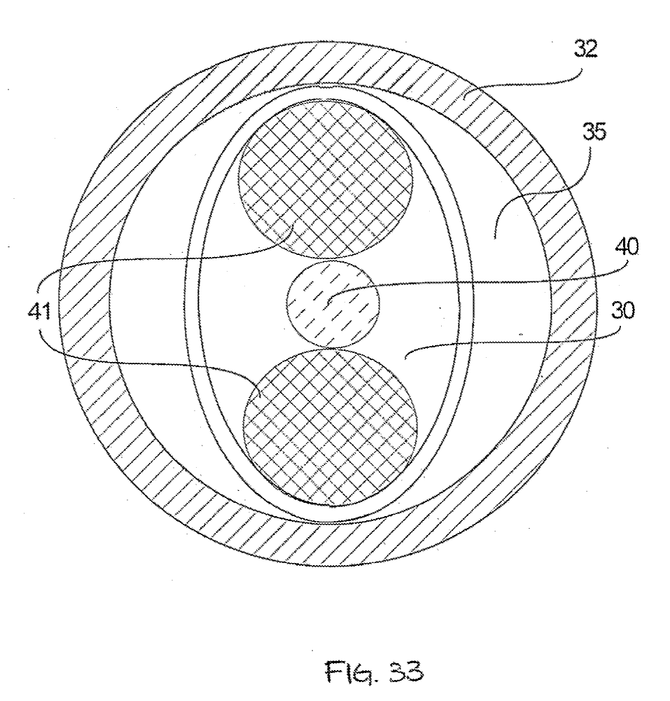

The valve prosthesis 10 and all subsequently described alternative embodiments are designed so that they can be delivered to the implantation site within a vessel or duct lumen using known endoluminal catheter techniques, as well as implanted by an open surgical procedure. Because the valve prosthesis 10 is comprised of a flexible deformable body 12, the valve prosthesis 10 could be transported through the lumen of a vessel 14 to the desired implantation site in a radially and/or longitudinally collapsed site. Upon successful endoluminal delivery of the valve prosthesis 10 to the desired implantation site, the valve prosthesis 10 could be expanded to its functional state using a variety of known endoluminal catheter techniques, including but not limited to, the use of an endoluminal catheter 40 with a plurality of inflatable balloons 41 at its distal aspect. A proposed method for endoluminal catheter implantation of the valve prosthesis 10 is depicted in FIGS. 32 and 33 for illustration purposes and not as a limitation. The catheter 40 depicted in FIG. 32 includes three balloons 41 at its distal aspect. The balloons 41 and 42 are inflatable and deflatable via a lumen in the catheter 40. The catheter 40 is inserted into the lumen of he vessel and the distal aspect of the catheter 40 (with the balloons 41 and 42) is positioned at the desired site of implantation with the balloons 41 and 42 deflated. Prior to implantation, the collapsed valve prosthesis 10 is coaxially surrounding the three deflated balloons 41 and 42. The larger balloon 42 is most distal and is generally cylindrical and/or conical in shape. The larger balloon 42 is designed to inflate to a diameter that approximates or slightly exceeds the diameter of the upstream portion of the valve prosthesis 10 (depending on the elasticity of the membrane material). When inflated, this larger balloon 42 will press the outer surface of the upstream portion of the conical deformable body 12, which can have an adhesive substance applied to it as previously described, against the inner surface of the vessel walls, thereby allowing the previously described circumferential area of attachment on the outer surface of the generally conical deformable body 12 to form a generally circumferential attachment to the vessel wall. Two smaller generally cylindrical or conical balloons 41 are located immediately proximal to the distal balloon in opposition to each other. When inflated, these small balloons 41 press the opposing areas of attachment of the downstream portion of the conical deformable body 12 to the inner surface of the vessel wall (as depicted in FIG. 14 and FIG. 15), allowing attachments to form between opposing areas of the downstream portion of the valve prosthesis 10 and the inner surface of the vessel wall. This proposed method of endoluminal catheter implantation is for illustration purposes and is not a limitation on implantation technique of the valve prosthesis 10. Similarly, a self-expanding wire frame could be extended from the distal aspect of a catheter 40 to exert pressure on the appropriate areas of the membrane to secure an attachment, and then retracted back into the catheter 40 once the attachment has been achieved. With these sample implantation methods, as an alternative to or in addition to adhesive susbstance(s), barbs (comprised of a metal, plastic or bioabsorbable material) which are incorporated into the valve prosthesis 10 projecting radially could be used to secure attachment of the valve prosthesis 10 to the vessel 14 by penetrating the vessel wall.

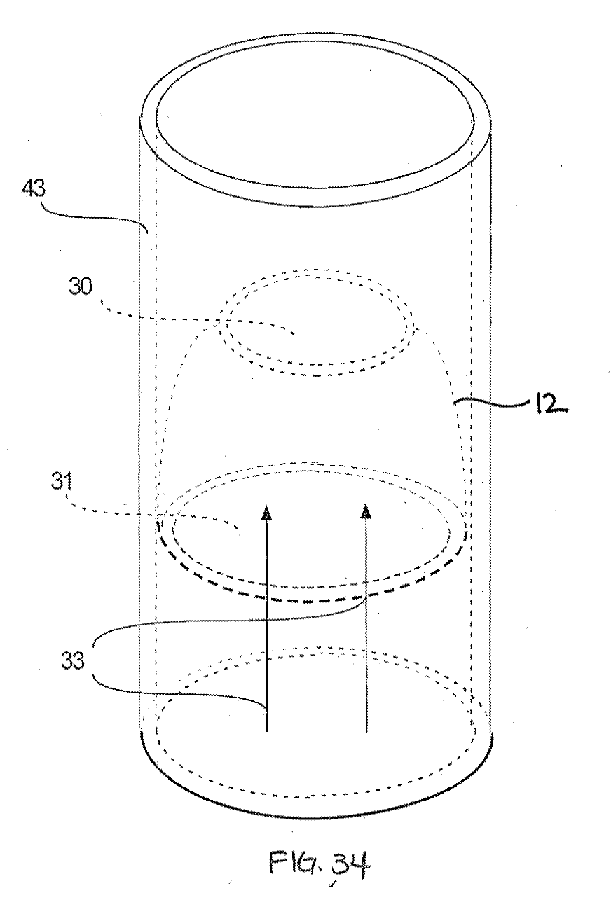

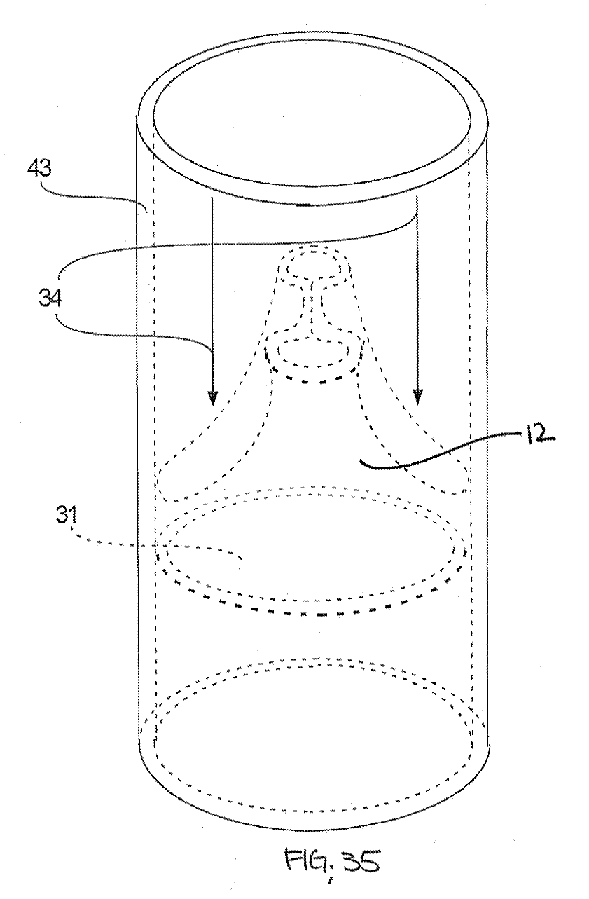

As depicted in FIGS. 34 and 35, an alternate embodiment of the valve prosthesis 10 includes the generally conical deformable body 12 within a conduit 43. The conduit 43 can be constructed from a material identical to that of the valve prosthesis 10 of the same of different thickness, or can be constructed from a different biocompatible synthetic or biological material (as previously described). In addition, the conduit 43 can be of non-uniform thickness itself, including one or more thicker regions to provide a support structure. As with the valve prosthesis 10 itself, the conduit 43 may also be treated and/or coated with any number of surface and/or material treatments and/or be partially or fully seeded with cultured tissues cells (e.g. endothelial cells) derived from either a donor or the host patient. The valve prosthesis 10 (i.e. generally conical deformable body 12) can be attached to the conduit with an adhesive substance or sutures or staples, or the entire combined valve and conduit structure be molded as a single piece of biocompatible material. The outer surface of the conduit can be attached to the inner surface of the vessel 14 using a variety of methods, including but not limited to the use of adhesive substances, barbs, sutures, or staples. Alternate embodiments can include a plurality of valves implanted serially within a conduit 43.

A variety of known endoluminal catheter methods can be used for implantation of the previously described embodiment consisting of a valve prosthesis 10 or a plurality of valve prosthesis 10 within a conduit 43. By way of example (and not as a limitation), the valve-conduit assembly could be delivered to the site of implantation in a radially and/or longitudinally collapsed state, with the collapsed valve-conduit assembly coaxially surrounding a plurality of deflated balloons at the distal aspect of the catheter. To implant the valve-conduit assembly, the balloons 41 and 42 can be inflated to bring the outer surface of the conduit in contact with the inner surface of the vessel 14, allowing adhesive substances and/or barbs (incorporated into the conduit and/or valve and projecting radially outward) to form attachment(s).

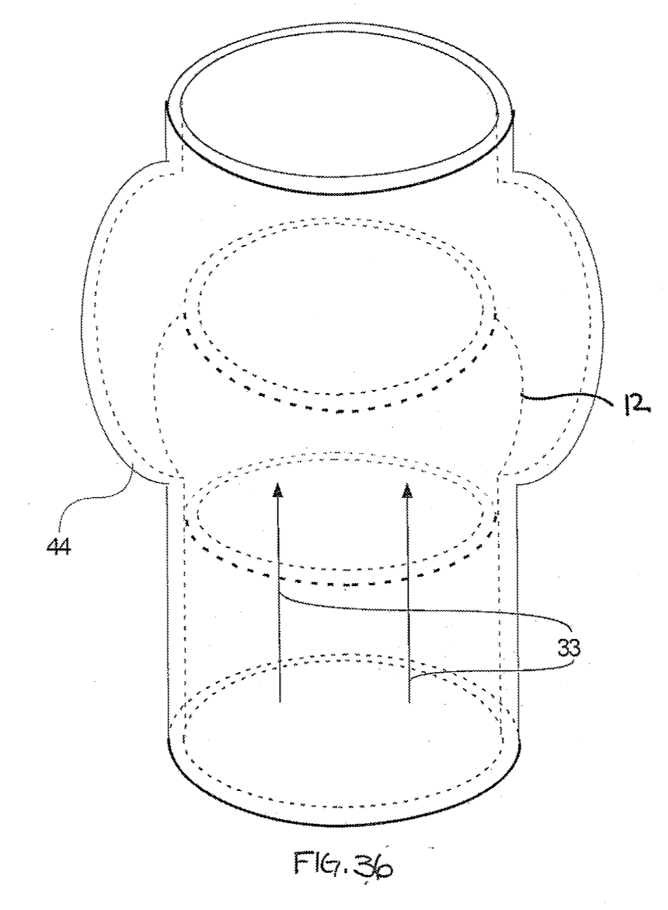

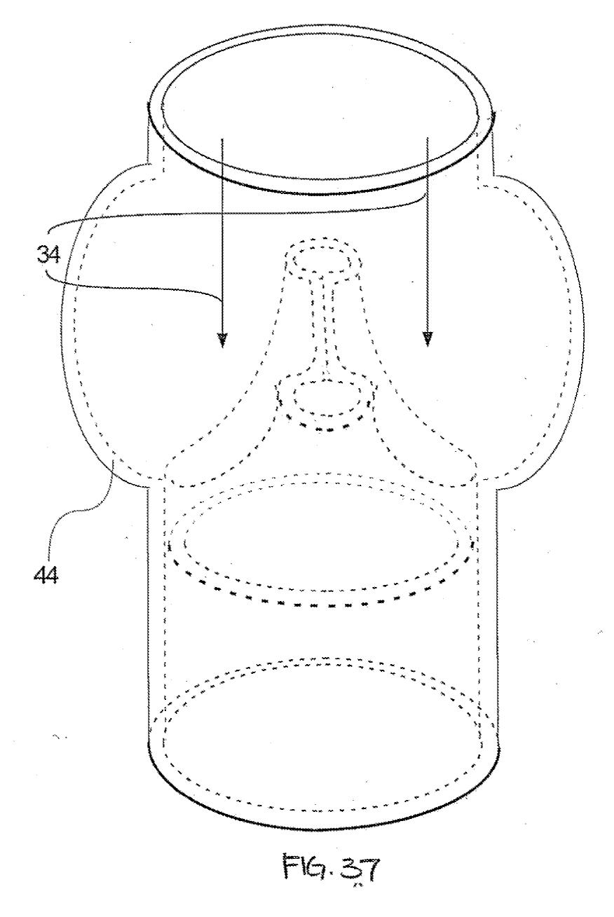

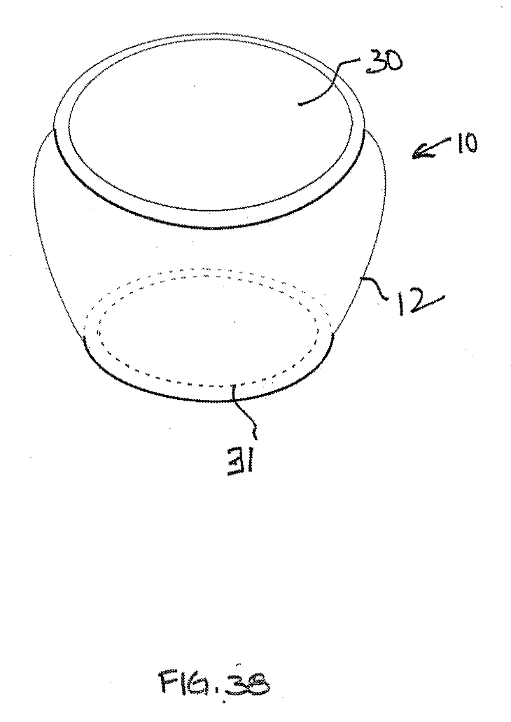

In an alternate embodiment depicted in FIGS. 36 and 37, the valve prosthesis 10 is within a conduit that contains in its mid-portion (in regards to the axial direction) a dilated portion 14, known as a complete sinus. The valve prosthesis 10 is positioned within the conduit 44, so that the downstream aspect of the valve prosthesis 10 is within the mid-portion of the complete sinus of the conduit 44 in the axial direction, and the upstream aspect of the valve prosthesis 10 is circumferentially attached to the non-dilated upstream portion of the conduit 44. Because the upstream aspect of the valve prosthesis 10 (i.e. generally conical deformable body 12) circumferentially attaches to the relatively smaller diameter non-dilated portion of the conduit 44, and the downstream aspect of the valve prosthesis 10 attaches to the relatively larger diameter mid-portion of the complete sinus, the diameter of the downstream valve opening 30 in this embodiment should be greater than the diameter of the upstream portion, but smaller than the diameter of the sinus. For clarification purposes, the valve portion of this embodiment is depicted without the conduit and sinus in FIG. 38. This is a generally conical deformable body 12 with the larger diameter opening 30 located downstream and the smaller diameter opening upstream 31. Alternatively, a generally cylindrical deformable body 12 could be employed as the valve prosthesis 10 in this embodiment if the diameter of the conduit's sinus was only slightly greater than the diameter of the remainder of the conduit 44. The key is that the maximum diameter of the downstream opening 30 of the valve prosthesis 10 be smaller than the maximum diameter of the mid-portion of the sinus, so that when attached as described previously an elliptical downstream opening 30 is formed and the outer surfaces of the valve leaflets are exposed to retrograde flow (and therefore will change to the closed position when acted upon by retrograde flow).

In an alternate embodiment, the entire conduit with complete sinus and the valve prosthesis 10 could be molded as a single structure instead of assembled from separate parts. Alternate embodiments can include a conduit with a plurality of complete sinuses, each sinus with a valve prosthesis 10.

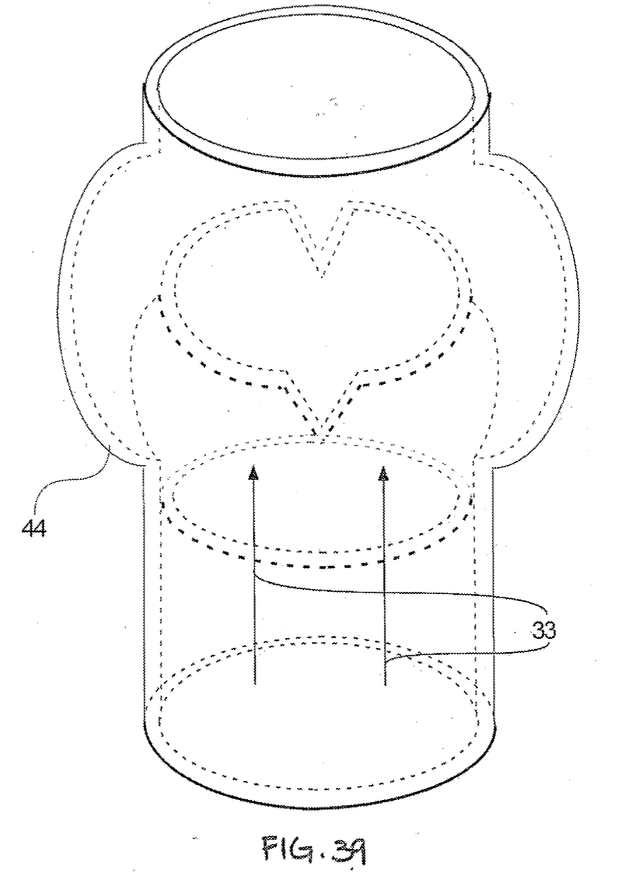

All of the features of the previously described embodiments can be used with a conduit with or without a sinus. For example, FIG. 39 depicts a valve prosthesis 10 with areas cut out of the downstream aspect of the valve prosthesis 10 (to completely restrict retrograde flow as previously described) within a conduit 44 with a full sinus.

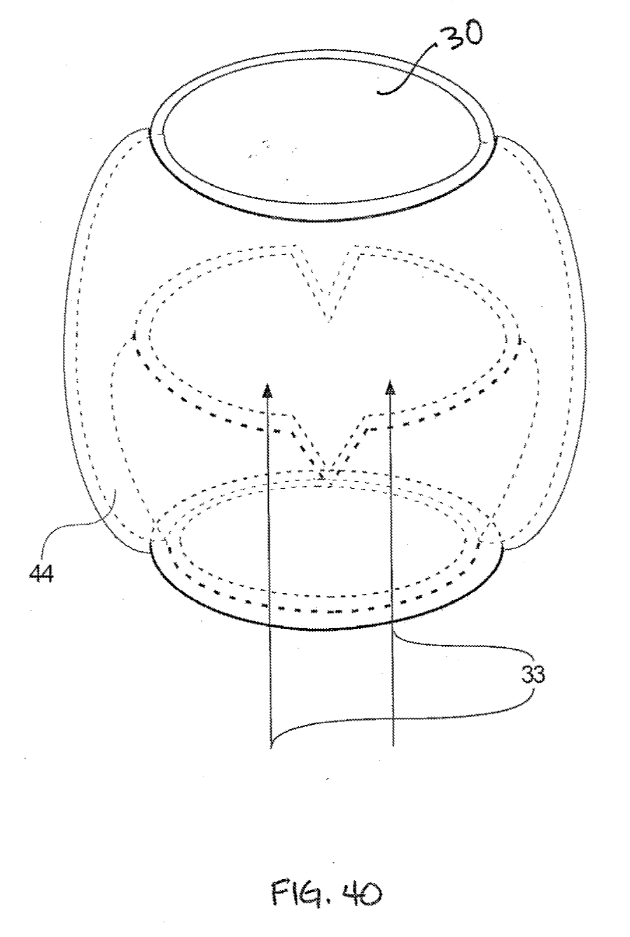

The lengths of the non-dilated portion(s) of a conduit 44 with a full sinus can be variable in length relative to the length of the full sinus itself. Alternately, the conduit 44 can consist only of a full sinus without any straight tubular portions, as depicted in FIG. 40.

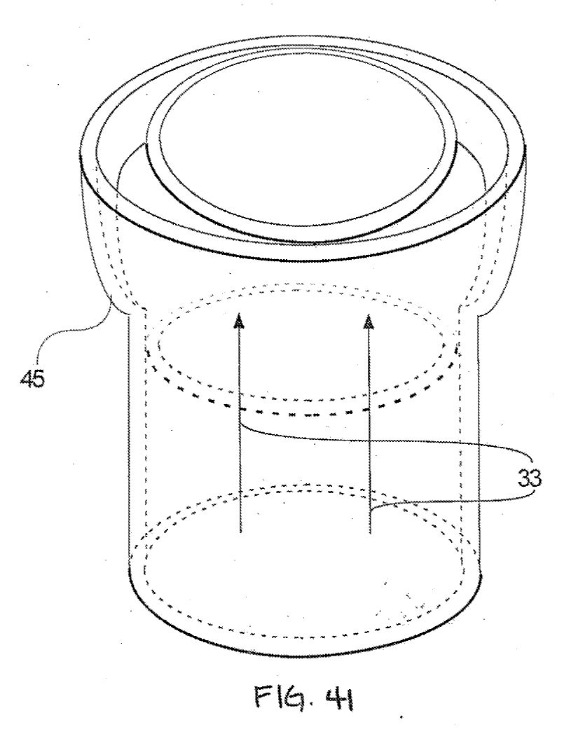

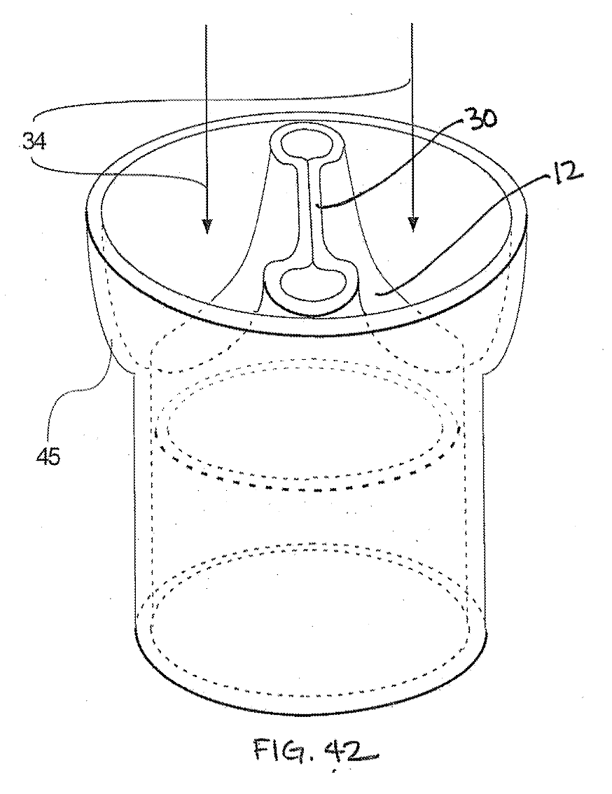

In an alternate embodiment depicted in FIGS. 41 and 42, the valve prosthesis 10 is within a conduit 45 that includes a dilated portion 45 at its downstream end, referred to in this document as a half-sinus (as opposed to the complete sinus depicted in FIGS. 36, 37 and 39. All of the features of the previously described embodiments can be used with a conduit with a half-sinus. In FIGS. 41 and 42, the half-sinus itself are generally conical in shape with the wall's bowing outward; however, the half sinus can also be generally conical shape with a sinusoidal or straight walls. Similarly, regarding embodiments which include a complete or half-sinus, the complete sinus can have a variety of configurations so long as a dilated segment of conduit is achieved and the downstream aspect of the valve prosthesis 10 is attached as previously described within the mid-portion of the dilated segment. The widest portion of the complete sinus or half-sinus, when viewed form above on cross section, can be generally round (as shown in the perspective views in FIGS. 36, 37, 39, 41 and 42), generally oval, generally square or diamond shaped, or generally rectangular.

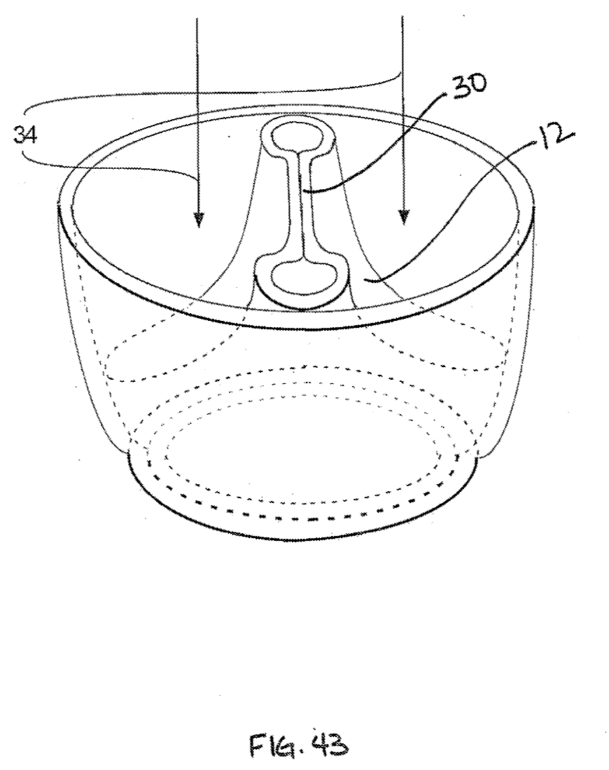

The axial length of the non-dilated portion of a conduit 45 with a half-sinus can be variable in length relative to the axial length of the half-sinus itself. Alternately, the conduit can consist only of a half sinus without any straight tubular portion, as depicted in FIG. 43.

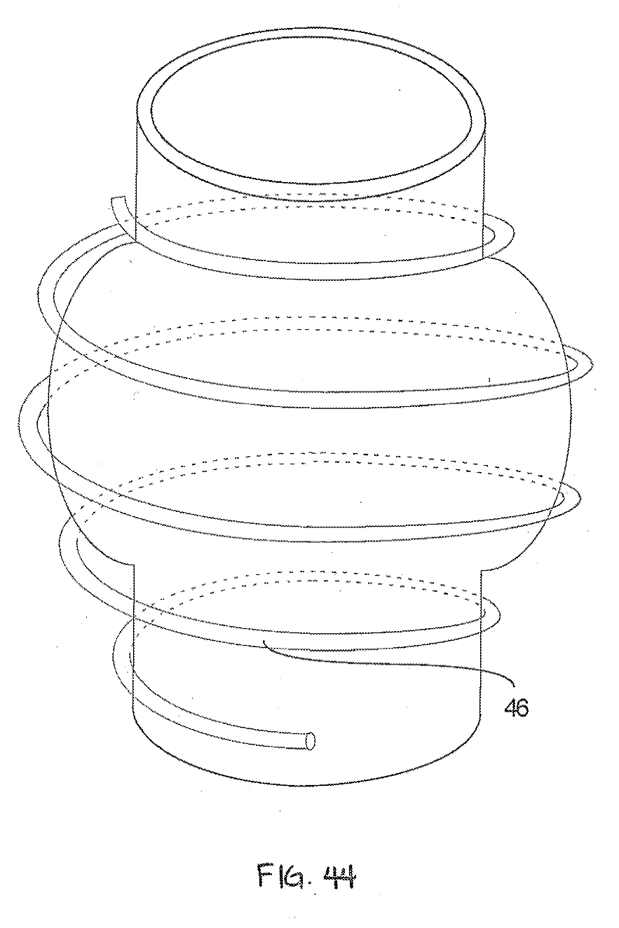

In alternate embodiments, a balloon-expandable or self-expanding stent 46 can be attached to the outer surface of all or part of the conduit (with a complete sinus or half-sinus or without a sinus) or incorporated within all or part of the material of the conduit (with a complete sinus or half-sinus or without a sinus) or attached to all or part of the inner surface of the conduit (with a complete sinus or half-sinus or without a sinus). Alternately, a balloon expandable stent or self-expanding stent 46 can be covered with the conduit membrane material both on the outer surface and on the inner surface. For conduits with a complete sinus or half sinus, a balloon-expandable or self-expanding stent 46 can serve to prevent the complete sinus or half-sinus from collapsing due to inward pressure from the vessel 14 that it is implanted in. The material used for the construction of these stents 46 can include, but is not limited to medical grade stainless steel, a special alloy of nickel and titanium called Nitinol, and bioabsorbable materials. A broad variety of stent designs can be employed as part of this invention, as are known or will be known to endovascular specialists in the medical community. FIG. 44 depicts a self-expanding coil-type Nitinol stent attached to the outside of a conduit with a complete sinus; the stent-conduit-valve assembly is in the implanted state. Possible stent designs which can be attached to or incorporated into a conduit as part of this invention include, but are not limited to, the Palmaz-Corinthian Stent, Palmaz-Schatz Stent, Wallstent, Bard Luminex Stent, Symphony Stent, S.M.A.R.T. Stent, Perflex Stent, AVE stent, AVE SE stent, Intrastent, Instent, Herculink stent, Mammotherm stent, and Dynalink stent designs. Methods for attaching part or all of the conduit to these frames or stents can include, but are not limited to, suturing, adhesive substances, and staples. In addition, portions of the valve prosthesis 10 itself (i.e. the generally conical shaped deformable body 12 within the conduit) can be attached to the frame or stent through the conduit using a variety of techniques, including but not limited to, sutures and/or staples. For embodiments of this invention in which a balloon expandable or self-expandable stent or frame is attached to or incorporated in the conduit material (including conduits with a complete sinus or sinuses, a half-sinus, or without a sinus), the stents or frames generally have two configurations. The first configuration is the unexpanded configuration, in which the stent or frame has a reduced diameter which facilitates advancement of the prosthesis though the lumen of the vessel 14, such as during percutaneous endoluminal delivery of the prosthesis to a point of treatment within the vessel 14. The second configuration is the expanded configuration, in which the stent or frame has an expanded diameter so that portions of the outer surface of the prosthesis interact with the inner surface of the vessel wall.

The method of stent-conduit-valve implantation depends on the stent design and stent material. For example, stent-conduit-valve assemblies with balloon-expandable stents can be coaxially mounted in the unexpanded state on deflated balloons at the distal aspect of an endoluminal catheter. The catheter 40 is used to position the stent-conduit-valve at the desired site of implantation. When stent-conduit-valve is at the desired site of implantation, the balloons 41 and 42 are inflated to expand the balloon-expandable stent-conduit-valve; this technique of endoluminal stent implantation is well-known to practitioners of the art. It is noted, however, that with this invention the stent-conduit-valve will have to mounted on the balloons 41 and 42 so that when the balloons 41 and 42 are inflated they do not destroy or deform the valve (i.e. generally conical deformable body 12) or valves within the conduit. In additions to the traditional balloon expansion technique for implantation, the stent and/or outer surface of the conduit can be attached to the vessel wall using an adhesive substance or substances, and/or the stent itself can include a plurality of barbs that project radically outward and on deployment penetrate the vessel wall to form attachments. When a self-expandable stent is attached to or incorporated into the conduit as part of the invention, the stent-conduit-valve is mounted in a collapsed state coaxially surrounding the distal aspect of the endoluminal catheter. The distal aspect of the catheter 40 includes a means to retain the stent-conduit-valve in the unexpanded state with a reduced radius and a means to release the stent-conduit-valve at the desired location. Upon release at the desired location, the self-expanding stent assumes the expanded, implanted state at the desired location within the lumen of the vessel 14. There are a variety of mechanisms to retain and release a self-expanding stent at the distal aspect of an endoluminal catheter 40; these mechanisms are known to practitioners of the art and any of these can be utilized with the valve prosthesis 10. For example, the valve prosthesis 10 can be retained in the unexpanded configuration within a sleeve at the distal aspect of the catheter 40, and then released by a mechanism which pushes the valve prosthesis 10 out of the sleeve.

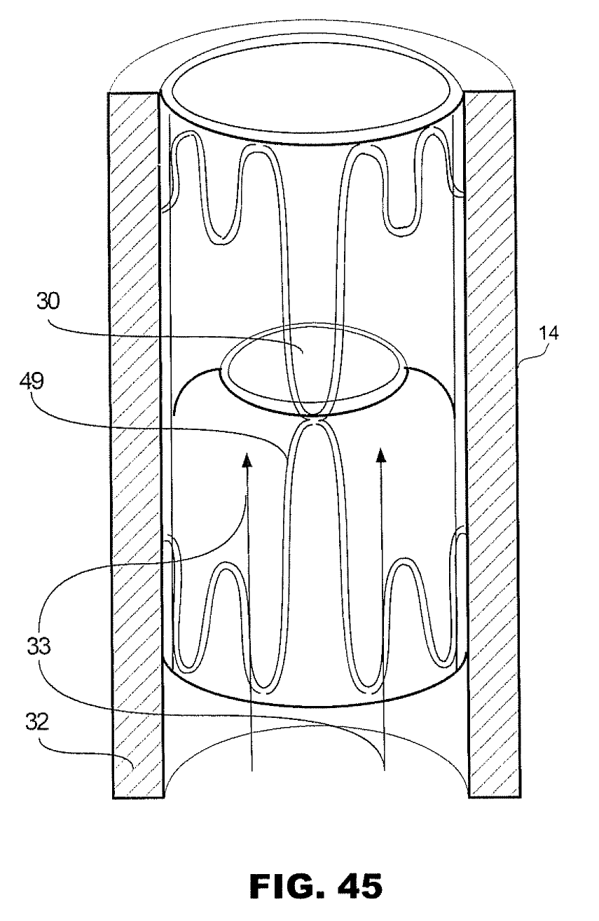

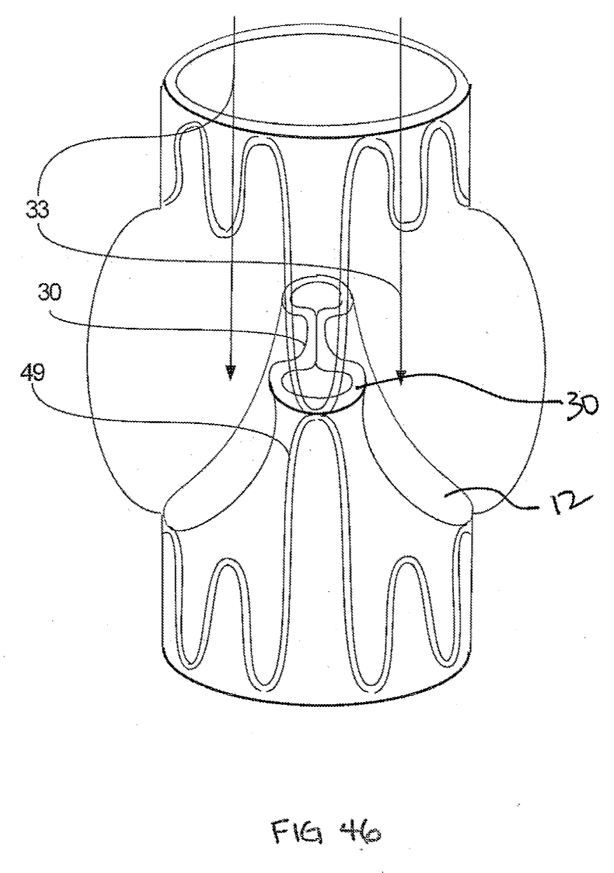

In another embodiment of this invention depicted in FIG. 45, the generally conical deformable body 12 and conduit is mounted in a self-expanding or balloon expandable stent 49. The self-expanding or expandable stent 49 consists of two cylindrical regions, one upstream and one downstream. The two cylindrical regions are joined by two opposing longitudinal struts. The conduit attaches circumferentially to either end of the stent 49 and to the longitudinal struts. The conduit contains a generally conical deformable body 12 as previously described; the circular upstream opening of the generally conical deformable body 12 attaches circumferentially to the upstream cylindrical portion of the stent 49 (through the conduit). The elliptical downstream opening 30 attaches the midportion of the two longitudinal opposing struts (through the conduit). The design of this stent 49 has gaps so as not to support the conduit walls behind the valve leaflets. This design allows for expansion of the elastic conduit deformable body 12 radially outward upon retrograde flow (FIG. 46), with the sinus area between the valve leaflets and the conduit collapsing again with antegrade flow (as shown in FIG. 45). Note that the stent 49 shown in the accompanying drawings in by way of example; a variety of stent 49 designs with two cylindrical portion joined by struts can be used to accomplish the same goal of allowing expansion and contraction of the sinuses with retrograde and antegrade flow respectively. As with the previously described embodiments which include a conduit and an expandable frame or stent, the stent 49 can be made from a variety of materials, including but not limited to stainless steel wire, Nitinol (an alloy of nickel and titanium), and various bioabsorbable materials. The stent 49 can be attached to the outer surface of the conduit, or incorporated into the conduit material, or attached to the inner surface of the conduit. The stent 49 can be attached to the conduit and the generally conical deformable body 12 using a variety of methods, including but not limited to sutures and/or adhesive substance(s) and/or staples.

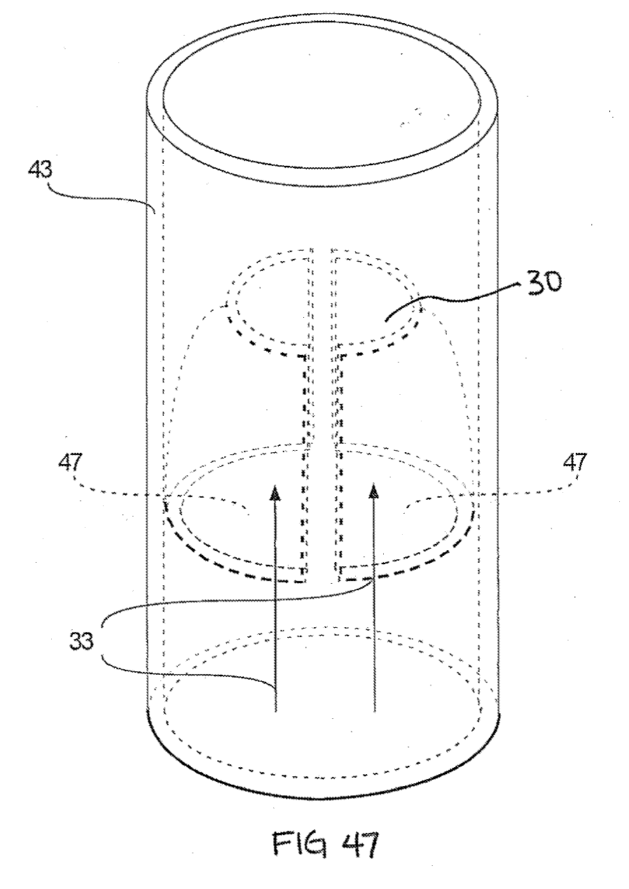

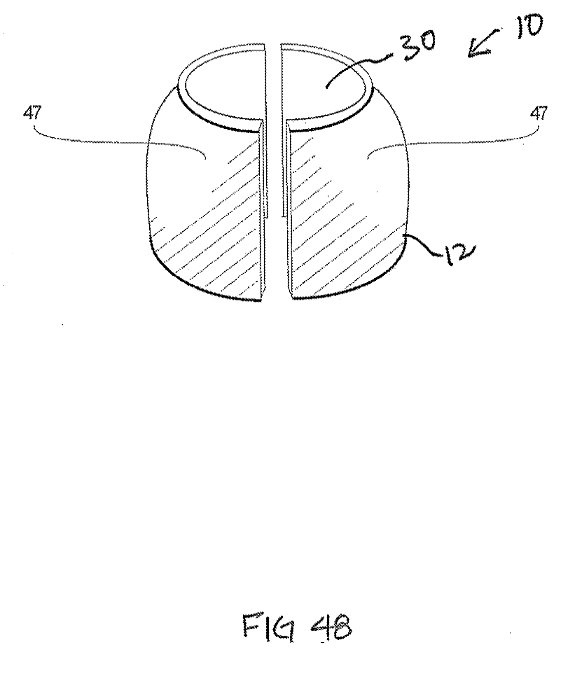

In another embodiment of this invention depicted in FIG. 47, two separate sheets of flexible membrane 47 are used to make a valve prosthesis 10 within a conduit 43. Each sheet of flexible membrane 47 is generally the shape of one half of the generally conical shaped deformable body 12 described in the aforementioned embodiments. The outer surface of each piece of flexible deformable body 12 is attached to the inner surface of the conduit in a manner similar to the previously described embodiments, as depicted in FIG. 48. There can be some variability in the shapes of the separate membrane pieces; one variant is depicted in FIG. 9. In other embodiments, a plurality of flexible membranes can be used to make a valve prosthesis 10 within a conduit, each in the shape of a faction of a generally conical deformable body 12. For example, three pieces of flexible membrane can be used to form a valve prosthesis 10, with each piece approximately one-third (divided in the axial direction) of the generally conical shaped deformable body 12 described in the aforementioned embodiments, to create a valve prosthesis 10 with three valve leaflets. Two or more pieces of flexible deformable body 12 can be used to form a valve prosthesis 10 in the manner described within a conduit without a sinus, within a conduit with a complete sinus, and within a half-sinus.

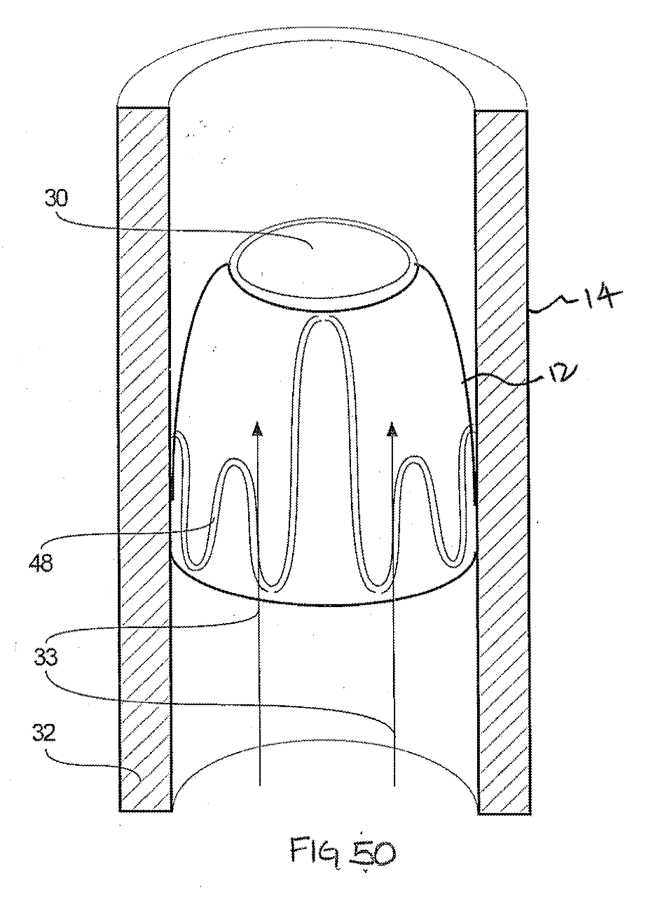

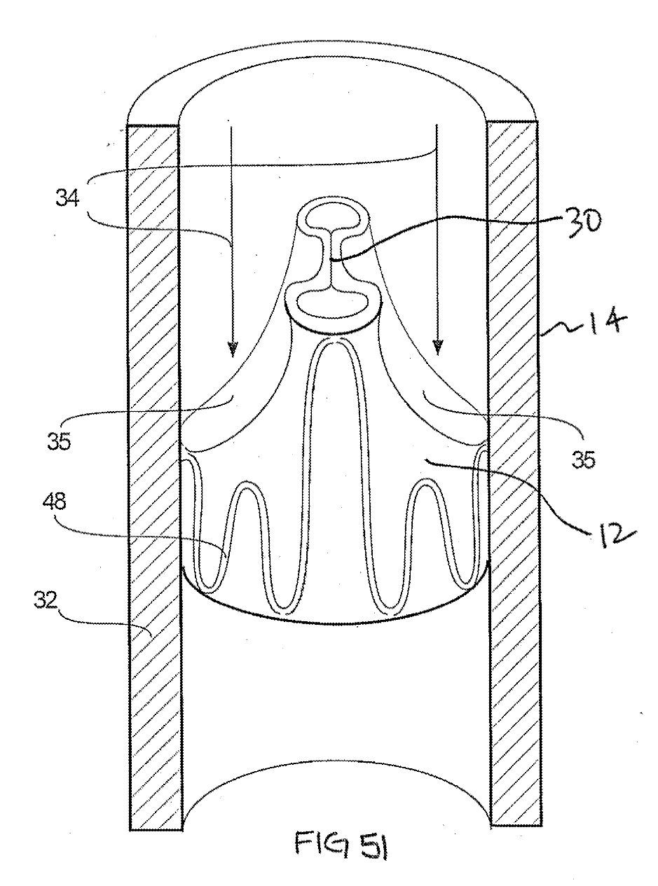

In an alternate embodiment depicted in FIGS. 50 and 51, a generally conical deformable body 12 can be attached directly to a balloon expandable frame or stent 48 the frame can be made form a variety of materials, including but not limited to stainless steel wire, Nitinol (an alloy) of nickel and titanium), and various bioabsorable materials. The frame 48 can be attached to the outer surface of the generally conical membrane, or incorporated into the membrane material, or attached to the inner surface of the generally conical deformable body 12. Alternately, separate layers of membrane material can be attached to the inner and outer surface of the frame 48 to enclose the frame 48. The frame 48 can be attached to generally conical deformable body 12 using a variety of methods, including but not limited to sutures and/or adhesive substance(s) and/or staples. The frame 48 is generally cylindrical or conical in shape, and circumferentially attached to the upstream aspect of the valve prosthesis 10. The frame 48 includes two struts in general opposing positions that extend axially to the downstream aspect of the generally opposing positions that extend axially to the downstream aspect of the generally conical deformable body 12. When the generally conical deformable body 12 is attached to the frame 48 and the frame 48 is in the expanded state, the generally conical deformable body 12 has generally round upstream opening 31, and a generally elliptical downstream opening 30 at the downstream when the valve prosthesis 10 is in the open state with antegrade fluid flow (as in previous embodiments). As with previous embodiments, the shape of the generally conical deformable body 12 when attached in this manner allows the outer surface of the valve leaflets to be exposed to retrograde flow, and therefore the valve will close when retrograde fluid flow exerts force on the outer surface of the leaflets as depicted in FIG. 50. The frame 48 depicted in FIGS. 50 and 51 is a curved zig zag design, but a variety of balloon expandable and self-expandable frame 48 design can be attached to a generally conical deformable body 12 in scope of this invention. When in the expandable configuration, the frame exerts a pressure outward against the vessel wall to secure the entire valve assembly in place. The attachment of the entire assembly consisting of a frame 48 generally conical deformable body 12 to the vessel wall can be supplemented by using an adhesive substance (or substances) on the all or portions of the outer surface of the generally conical deformable body 12 and/or the expandable frame 48. Alternately, the frame 48 can include a plurality of barbs that extend radially outward to penetrate the vessel wall and help secure the attachment.

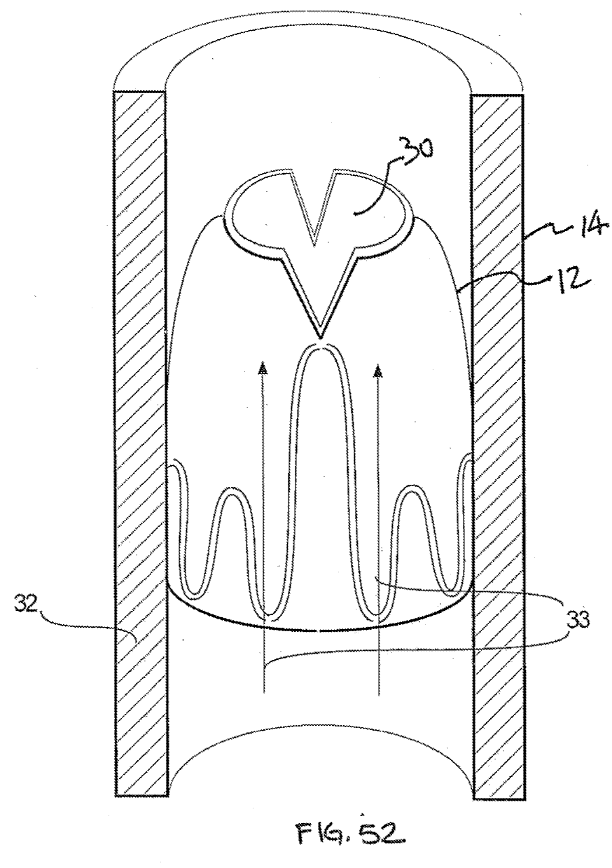

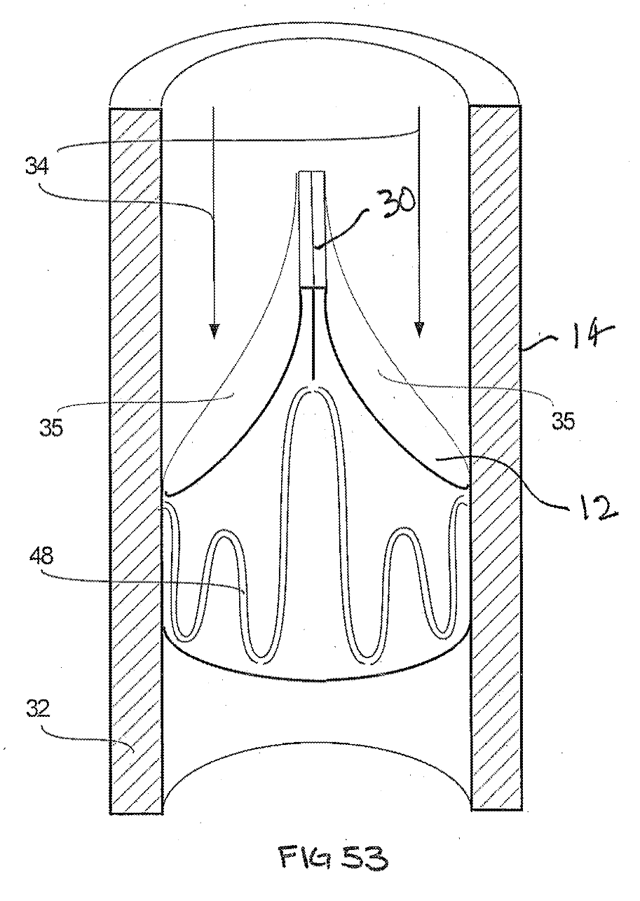

The embodiment pictured in FIGS. 50 and 51 includes a generally conical deformable body 12 with walls bowed outwards. However, an expandable frame 48 can be combined in the manner described with any of the features of other embodiments described in this application. For example, FIGS. 52 and 53 depicts a frame attached to the outside of a generally conical deformable body 12 with triangle-shaped pieces cut out of the downstream aspect of the deformable body 12; these opposing cut out pieces are oriented over the opposing struts a shown. As discussed previously, the exact shape of the opposing cut out areas can vary within the scope of the valve prosthesis 10. Also, as described previously, the downstream edges of the valve leaflets can be flat, crowned or scalloped.

The valve prosthesis 10 can include one or more radiopaque markers, attached to or coated onto one or more locations along the valve. The position of the one or more radiopaque markers can be selected so as to provide information on the position and orientation of the valve prosthesis during and subsequent to implantation. Included in the scope is the positioning of radiopaque and/or sonographic markers on the valve leaflets themselves, so that valve functionality can be confirmed radiographically and/or sonographically during and/or following implantation. In addition, he various edges of the valve and/or conduit can be tapered in an effort to minimize the transition between surface (such as the transition between inner surface of the vessel 14 and inner surface of the valve prosthesis 10 between the inner surface of the conduit and the inner surface of the valve prosthesis 10 and/or the inner surface of the vessel 14 and the inner surface of the conduit.

Although the embodiments described above demonstrates two-leaflets (bicuspid), design employing a different number of valve leaflets (e.g., three leaflet) are possible. Although the embodiments described above have symmetric leaflets, leaflets of different sizes and configurations can be used in conjunction with one anther by varying the attachment points of the proximal (downstream) portion of the device to the vessel wall. As previously stated, all of the features of the described embodiments can be combined in varying combinations within the scope of this invention.

While the present invention has been shown and described in detail above, it will be clear to the person skilled in the art that changes and modifications may be made without departing from the spirit and scope of the invention. As such, that which is set forth in the foregoing description and accompanying drawings is offered by way of illustration only and not as limitations.

When introducing elements of aspects of the invention or the embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

As various changes could be made in the above constructions, products, and methods without departing from the scope of aspects of the invention, it is intended that all matter contained in the above description and shown in the accompanying drawings shall be interpreted as illustrative and not in a limiting sense.

What is claimed is:

1. An implantable valve prosthesis (10) comprising a deformable body (12) having a first configuration that permits fluid flow communication in one direction while a second configuration prevents fluid communication in an opposite direction. The deformable body (12) defines a generally cylindrical configuration with a downstream opening in communication with an opposing upstream opening such that when the deformable body (12) is in the first configuration the downstream opening has substantially the same shape as the upstream opening, and when the deformable body (12) is in the second configuration the downstream opening has a smaller shape than the upstream opening, thereby preventing fluid flow communication in the opposite direction.

2. The implantable valve prosthesis (10) of claim 1, wherein the deformable body (12) is made from two or more membranes.

3. The implantable valve prosthesis (10) of claim 1, wherein the downstream opening (30) in the first configuration is in an open position that permits fluid flow in one direction only and in the second configuration the downstream opening (3) is in a closed position such that retrograde fluid flow through the downstream opening to the upstream opening is prevented in the second configuration.

4. The implantable valve prosthesis (10) of claim 1 wherein the deformable body (12) is bowed outwardly in the second configuration.

5. The implantable valve prosthesis (10) of claim 1, wherein the deformable body (12) is adapted to engage a stent for retaining the prosthesis (10) inside the lumen of a vessel.

6. The implantable valve prosthesis (10) of claim 1, wherein the deformable body (12) defines a scalloped opening.

7. The implantable valve prosthesis (10) of claim 1, wherein the deformable body (12) defines opposing slots along the downstream opening (30).

8. The implantable valve prosthesis (10) of claim 1, wherein the deformable body (12) is retained inside the lumen of a vessel using by use of an adhesive.

9. The implantable valve prosthesis (10) of claim 1, wherein the deformable body (12) is adapted to be engaged to a catheter (40) for insertion into the lumen of a vessel.

10. The implantable valve prosthesis (10) of claim 1, wherein the deformable body (12) has a conical configuration.

11. The implantable valve prosthesis (10) of claim 1, wherein the deformable body (12) has a cylindrical configuration.

12. The implantable valve prosthesis (10) of claim 1, wherein the deformable body (12) includes one or more expandable balloons (41, 42).

13. An implantable valve prosthesis (10) may include a deformable body (12) having a first position for permitting fluid flow communication inside a lumen in one direction only and a second position for preventing fluid flow communication inside the lumen in the opposite direction, the deformable body (12) being adapted to engage an expandable stent.

14. A method for deploying an implantable valve prosthesis (10) may include the steps of:
providing a catheter (40) defining a proximal end and a distal end;
attaching the distal end of the catheter (40) to an implantable valve prosthesis (10) having a deformable body (12) having a first configuration that permits fluid flow communication in one direction while a second configuration prevents fluid communication in an opposite direction with the deformable body (12) defining a generally cylindrical configuration with a downstream opening in communication with an opposing upstream opening such that when the deformable body (12) is in the first configuration the downstream opening has substantially the same shape as the upstream opening, and when the deformable body (12) is in the second configuration the downstream opening has a smaller shape than the upstream opening, thereby preventing fluid flow communication in the opposite direction; and
implanting the distal end of the catheter (40) inside the lumen of a body such that the implantable valve prosthesis (10) is disposed across the lumen of the body in a manner that permits selective fluid flow communication through the lumen by the deformable body of the implantable valve prosthesis (10).
